(19) United States
(12) Patent Application Publication
(10) Pub. No.: US 2021/0275503 A1
(43) Pub. Date: Sep. 9, 2021

(54) TARGETING HOMOLOGOUS RECOMBINATION: A NEW SYTHETIC LETHAL THERAPEUTIC PARADIGM

(71) Applicant: Cyteir Therapeutics, Inc., Lexington, MA (US)

(72) Inventors: Thomas O'SHEA, Wellesley, MA (US); Markus RENSCHLER, Lexington, MA (US); Todd BOWSER, Charlton, MA (US); Jean-Marc LAPIERRE, Pelham, NH (US); Kevin MILLS, Boxborough, MA (US)

(21) Appl. No.: 17/191,220

(22) Filed: Mar. 3, 2021

Related U.S. Application Data
(60) Provisional application No. 63/148,683, filed on Feb. 12, 2021.
(60) Provisional application No. 62/984,765, filed on Mar. 3, 2020.

(51) Int. Cl. A61K 31/426; A61P 35/00

(52) U.S. Cl. CPC A61K 31/426; A61P 35/00

(57) ABSTRACT

This application is directed to inhibitors of RAD51 represented by the following structural formula,

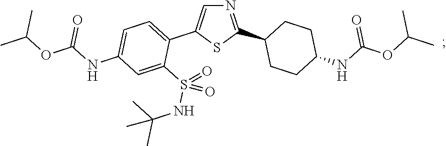 [SMILES: CC(C)OC(=O)Nc1ccc(-c2cnc([C@H]3CC[C@H](NC(=O)OC(C)C)CC3)s2)c(S(=O)(=O)NC(C)(C)C)c1]

and methods for its use, such as to treat cancer.

### RELATED APPLICATIONS

This application claims priority to, and the benefit of, U.S. Provisional Application No. 62/984,765, filed on Mar. 3, 2020, and 63/148,683, filed on Feb. 12, 2021, the contents of each of which are incorporated herein by reference in their entirety.

### BACKGROUND

RAD51 is a eukaryote gene. The protein encoded by this gene is a member of the RAD51 protein family which assists in repair of DNA double strand breaks. RAD51 family members are homologous to the bacterial RecA, Archaeal RadA and yeast RAD51. The protein is highly conserved in most eukaryotes, from yeast to humans. In humans, RAD51 is a 339-amino acid protein that plays a major role in homologous recombination of DNA during double strand break (DSB) repair. RAD51 catalyzes strand transfer between a broken sequence and its undamaged homologue to allow re-synthesis of the damaged region.

Studies have demonstrated sensitization to certain DNA damaging therapies associated with cellular defects in proteins that promote HR DNA repair. This sensitization is particularly dramatic for DNA cross-linking chemotherapeutic drugs (30-100 times) and ionizing radiation (3-5 times) (Godthelp et al., Nucleic Acids Res., 30:2172-2182, 2002; Tebbs et al., Proc. Natl. Acad. Sci. USA, 92:6354-6358, 1995; Takata et al., Mol. Cell Biol., 21:2858-2866, 2001; Liu et al., Mol. Cell, 1:783-793, 1998). Several groups have recently demonstrated that HR can be partially inhibited in order to sensitize cells to DNA damaging therapies. Inhibition of XRCC3 (a RAD51 paralog protein) has been demonstrated using a synthetic peptide corresponding to another paralog protein. This peptide sensitized Chinese Hamster Ovary (CHO) cells to cisplatin and inhibited the formation of sub-nuclear RAD51 foci in response to DNA damage (Connell et al., Cancer Res., 64:3002-3005, 2004). Other researchers have inhibited the expression of the RAD51 protein itself (Russell et al., Cancer Res., 63:7377-7383, 2003; Hansen et al., Int. J. Cancer, 105:472-479, 2003; Ohnishi et al., Biochem. Biophys. Res. Commun., 245:319-324, 1998; Ito et al., J. Gene Med., 7(8):1044-1052, 2005; Collins et al., Nucleic Acids Res., 29:1534-1538, 2001) or blocked its function by over-expressing a dominant negative BRC peptide fragment derived from BRCA2 (Chen et al., J. Biol. Chem., 274:32931-32935, 1999). In view of the connection between increased sensitivity to certain DNA damaging therapies and cellular defects in HR DNA repair-related proteins, there is a need for additional compounds that inhibit RAD51.

While AID expression is normally transient and restricted to activated B-cells, several cancers show constitutive, ectopic AID expression including pancreatic cancer. AID is a promiscuous DNA damaging enzyme that targets widespread locations throughout the genome, leading to high levels of DNA replication stress. AID expressing cells become critically dependent on the homologous recombination factor RAD51 to survive this DNA replication stress.

### SUMMARY

In one aspect, the present disclosure provides a method of treating a cancer, the method comprising administering Compound 67A:

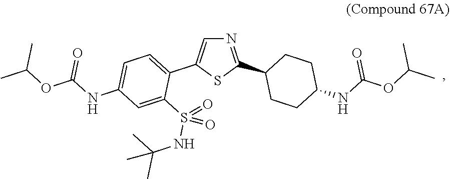 [SMILES: CC(C)OC(=O)Nc1ccc(-c2cnc([C@H]3CC[C@H](NC(=O)OC(C)C)CC3)s2)c(S(=O)(=O)NC(C)(C)C)c1]

or a pharmaceutically acceptable salt thereof, to a subject in need thereof at a dosage disclosed herein.

In one aspect, the present disclosure provides a method of treating a cancer, the method comprising administering a Compound 67A, to a subject in need thereof at a dosage disclosed herein.

In one aspect, the present disclosure provides a method of treating a cancer, the method comprising administering a composition comprising Compound 67A or a pharmaceutically acceptable salt thereof, to a subject in need thereof at a dosage disclosed herein.

In one aspect, the present disclosure provides a composition comprising Compound 67A or a pharmaceutically acceptable salt thereof for use in treating a cancer in a subject in need thereof at a dosage disclosed herein.

In one aspect, the present disclosure provides Compound 67A or a pharmaceutically acceptable salt thereof for use in treating a cancer in a subject in need thereof at a dosage disclosed herein.

In one aspect, the present disclosure provides Compound 67A for use in treating a cancer in a subject in need thereof at a dosage disclosed herein.

In one aspect, described herein is use of a composition (e.g., a composition comprising Compound 67A or a pharmaceutically acceptable salt thereof) in the manufacture of a medicament for the treatment of a cancer in a subject in need thereof at a dosage disclosed herein.

In one aspect, described herein is use of Compound 67A or a pharmaceutically acceptable salt thereof in the manufacture of a medicament for the treatment of a cancer in a subject in need thereof at a dosage disclosed herein.

In one aspect, described herein is use of Compound 67A in the manufacture of a medicament for the treatment of a cancer in a subject in need thereof at a dosage disclosed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows Compound 67A toxicology study design in rat and dog.

FIGS. 2A-2B show Compound 67A C1D15/22 human pharmacokinetics, wherein FIG. 2A shows the pharmacokinetic profile on cycle 1 for day 15 and 22 and FIG. 2B shows plasma AUC₂₄ vs dosage.

FIG. 3 shows Compound 67A administration at 90 mg QD on food effect pharmacokinetics at day 1 of cycle 1 in human subjects.

FIG. 4 shows Compound 67A phase 1 and 2 monotherapy trial protocol.

FIG. 5 shows response with metabolic response for a subject with diffuse large B-cell lymphoma administered Compound 67A.

FIG. 6 shows response in a subject with follicular lymphoma administered Compound 67A at 45 mg PO BID.

FIG. 7 shows response in a subject with CD⁺ follicular lymphoma administered Compound 67A at 30 mg BID for two cycles.

FIG. 8 shows response in a subject with myxofibrosarcoma administered Compound 67A.

FIG. 9 shows response in a subject with ovarian cancer administered Compound 67A.

### DETAILED DESCRIPTION

The compound herein is an effective RAD51 inhibitor for use in treating a cancer. The RAD51 inhibitor of the present disclosure inhibits homologous recombination by altering the nucleocytoplasmic distribution of RAD51 following DNA damage induction. Without wishing to be bound by theory, the RAD51 inhibitor of the present disclosure reduces the repair of AID-induced DNA double strand breaks, leading to AID-dependent cytotoxicity in both normal and malignant cells.

In some embodiments, the present disclosure provides a method of treating a cancer comprising administering to a subject in need thereof Compound 67A

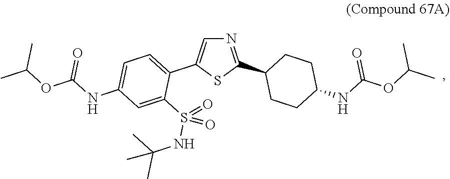 [SMILES: CC(C)OC(=O)Nc1ccc(-c2cnc([C@H]3CC[C@H](NC(=O)OC(C)C)CC3)s2)c(S(=O)(=O)NC(C)(C)C)c1]

or a pharmaceutically acceptable salt thereof, at a dosage from about 15 mg to about 700 mg.

In some embodiments, the present disclosure provides a method of treating a cancer comprising administering to a subject in need thereof Compound 67A at a dosage from about 15 mg to about 700 mg.

In some embodiments, the present disclosure provides a method of treating a cancer comprising administering to a subject in need thereof a composition comprising Compound 67A or a pharmaceutically acceptable salt thereof, at a dosage from about 15 mg to about 700 mg.

In some embodiments, the present disclosure provides a composition comprising Compound 67A, or a pharmaceutically acceptable salt thereof, for use in treating cancer in a subject in need thereof at a dosage from about 15 mg to about 700 mg.

In some embodiments, the present disclosure provides Compound 67A, or a pharmaceutically acceptable salt thereof, for use in treating cancer in a subject in need thereof at a dosage from about 15 mg to about 700 mg.

In some embodiments, the present disclosure provides Compound 67A for use in treating cancer in a subject in need thereof at a dosage from about 15 mg to about 700 mg.

In some embodiments, the present disclosure provides use of a composition comprising Compound 67A, or a pharmaceutically acceptable salt thereof, for the treatment of cancer in a subject in need thereof at a dosage from about 15 mg to about 700 mg.

In some embodiments, the present disclosure provides use of Compound 67A, or a pharmaceutically acceptable salt thereof, for the treatment of cancer in a subject in need thereof at a dosage from about 15 mg to about 700 mg.

In some embodiments, the present disclosure provides use of Compound 67A for the treatment of cancer in a subject in need thereof at a dosage from about 15 mg to about 700 mg.

In some embodiments, the present disclosure provides use of a composition comprising Compound 67A, or a pharmaceutically acceptable salt thereof, in the manufacture of a medicament for the treatment of cancer in a subject in need thereof at a dosage from about 15 mg to about 700 mg.

In some embodiments, the present disclosure provides the use of Compound 67A, or a pharmaceutically acceptable salt thereof, in the manufacture of a medicament for the treatment of cancer in a subject in need thereof at a dosage from about 15 mg to about 700 mg.

In some embodiments, the present disclosure provides the use of Compound 67A in the manufacture of a medicament for the treatment of cancer in a subject in need thereof at a dosage from about 15 mg to about 700 mg.

In some embodiments, the subject is a mammal.

In some embodiments, the mammal is human.

In some embodiments, the mammal is rat.

In some embodiments, the mammal is dog.

In some embodiments, Compound 67A is administered at a dosage of about 15 mg.

In some embodiments, Compound 67A is administered at a dosage of about 20 mg.

In some embodiments, Compound 67A is administered at a dosage of about 25 mg.

In some embodiments, Compound 67A is administered at a dosage of about 30 mg.

In some embodiments, Compound 67A is administered at a dosage of about 35 mg.

In some embodiments, Compound 67A is administered at a dosage of about 40 mg.

In some embodiments, Compound 67A is administered at a dosage of about 45 mg.

In some embodiments, Compound 67A is administered at a dosage of about 50 mg.

In some embodiments, Compound 67A is administered at a dosage of about 55 mg.

In some embodiments, Compound 67A is administered at a dosage of about 60 mg.

In some embodiments, Compound 67A is administered at a dosage of about 65 mg.

In some embodiments, Compound 67A is administered at a dosage of about 70 mg.

In some embodiments, Compound 67A is administered at a dosage of about 75 mg.

In some embodiments, Compound 67A is administered at a dosage of about 80 mg.

In some embodiments, Compound 67A is administered at a dosage of about 85 mg.

In some embodiments, Compound 67A is administered at a dosage of about 90 mg.

In some embodiments, Compound 67A is administered at a dosage of about 95 mg.

In some embodiments, Compound 67A is administered at a dosage of about 100 mg.

In some embodiments, Compound 67A is administered at a dosage of about 110 mg.

In some embodiments, Compound 67A is administered at a dosage of about 120 mg.

In some embodiments, Compound 67A is administered at a dosage of about 130 mg.

In some embodiments, Compound 67A is administered at a dosage of about 140 mg.

In some embodiments, Compound 67A is administered at a dosage of about 150 mg.

In some embodiments, Compound 67A is administered at a dosage of about 160 mg.

In some embodiments, Compound 67A is administered at a dosage of about 170 mg.

In some embodiments, Compound 67A is administered at a dosage of about 180 mg.

In some embodiments, Compound 67A is administered at a dosage of about 190 mg.

In some embodiments, Compound 67A is administered at a dosage of about 200 mg.

In some embodiments, Compound 67A is administered at a dosage of about 220 mg.

In some embodiments, Compound 67A is administered at a dosage of about 240 mg.

In some embodiments, Compound 67A is administered at a dosage of about 260 mg.

In some embodiments, Compound 67A is administered at a dosage of about 280 mg.

In some embodiments, Compound 67A is administered at a dosage of about 300 mg.

In some embodiments, Compound 67A is administered at a dosage of about 320 mg.

In some embodiments, Compound 67A is administered at a dosage of about 340 mg.

In some embodiments, Compound 67A is administered at a dosage of about 360 mg.

In some embodiments, Compound 67A is administered at a dosage of about 380 mg.

In some embodiments, Compound 67A is administered at a dosage of about 400 mg.

In some embodiments, Compound 67A is administered at a dosage of about 420 mg.

In some embodiments, Compound 67A is administered at a dosage of about 440 mg.

In some embodiments, Compound 67A is administered at a dosage of about 460 mg.

In some embodiments, Compound 67A is administered at a dosage of about 480 mg.

In some embodiments, Compound 67A is administered at a dosage of about 500 mg.

In some embodiments, Compound 67A is administered at a dosage of about 520 mg.

In some embodiments, Compound 67A is administered at a dosage of about 540 mg.

In some embodiments, Compound 67A is administered at a dosage of about 560 mg.

In some embodiments, Compound 67A is administered at a dosage of about 580 mg.

In some embodiments, Compound 67A is administered at a dosage of about 600 mg.

In some embodiments, Compound 67A is administered at a dosage of about 620 mg.

In some embodiments, Compound 67A is administered at a dosage of about 640 mg.

In some embodiments, Compound 67A is administered at a dosage of about 660 mg.

In some embodiments, Compound 67A is administered at a dosage of about 680 mg.

In some embodiments, Compound 67A is administered at a dosage of about 700 mg.

In some embodiments, Compound 67A is administered at a dosage of about 15±2 mg, 15±1.8 mg, 15±1.6 mg, 15±1.5 mg, 15±1.4 mg, 15±1.3 mg, 15±1.2 mg, 15±1.1 mg, 15±1 mg, 15±0.9 mg, 15±0.8 mg, 15±0.7 mg, 15±0.6 mg, 15±0.5 mg, 15±0.4 mg, 15±0.3 mg, 15±0.2 mg, or 15±0.1 mg.

In some embodiments, Compound 67A is administered at a dosage of about 20±2 mg, 20±1.8 mg, 20±1.6 mg, 20±1.5 mg, 20±1.4 mg, 15±1.3 mg, 20±1.2 mg, 20±1.1 mg, 20±1 mg, 20±0.9 mg, 20±0.8 mg, 20±0.7 mg, 20±0.6 mg, 20±0.5 mg, 20±0.4 mg, 20±0.3 mg, 20±0.2 mg, or 20±0.1 mg.

In some embodiments, Compound 67A is administered at a dosage of about 30±2 mg, 30±1.8 mg, 30±1.6 mg, 30±1.5 mg, 30±1.4 mg, 30±1.3 mg, 30±1.2 mg, 30±1.1 mg, 30±1 mg, 30±0.9 mg, 30±0.8 mg, 30±0.7 mg, 30±0.6 mg, 30±0.5 mg, 30±0.4 mg, 30±0.3 mg, 30±0.2 mg, or 30±0.1 mg.

In some embodiments, Compound 67A is administered at a dosage of about 45±2 mg, 45±1.8 mg, 45±1.6 mg, 45±1.5 mg, 45±1.4 mg, 45±1.3 mg, 45±1.2 mg, 45±1.1 mg, 45±1 mg, 45±0.9 mg, 45±0.8 mg, 45±0.7 mg, 45±0.6 mg, 45±0.5 mg, 45±0.4 mg, 45±0.3 mg, 45±0.2 mg, or 45±0.1 mg.

In some embodiments, Compound 67A is administered at a dosage of about 90±5 mg, 90±4.5 mg, 90±4 mg, 90±3.5 mg, 90±3 mg, 90±2.5 mg, 90±2 mg, 90±1.5 mg, 90±1 mg, 90±0.9 mg, 90±0.8 mg, 90±0.7 mg, 90±0.6 mg, 90±0.5 mg, 90±0.4 mg, 90±0.3 mg, 90±0.2 mg, or 90±0.1 mg.

In some embodiments, Compound 67A is administered at a dosage of about 130±10 mg, 130±9 mg, 130±8 mg, 130±7 mg, 130±6 mg, 130±5 mg, 130±4.5 mg, 130±4.0 mg, 130±3.5 mg, 130±3.0 mg, 130±2.5 mg, 130±2.0 mg, 130±1.5 mg, 130±1.0 mg, 130±0.9 mg, 130±0.8 mg, 130±0.7 mg, 130±0.6 mg, 130±0.5 mg, 130±0.4 mg, 130±0.3 mg, 130±0.2 mg, or 130±0.1 mg.

In some embodiments, Compound 67A is administered at a dosage of about 200±10 mg, 200±9 mg, 200±8 mg, 200±7 mg, 200±6 mg, 200±5 mg, 200±4.5 mg, 200±4.0 mg, 200±3.5 mg, 200±3.0 mg, 200±2.5 mg, 200±2.0 mg, 200±1.5 mg, 200±1.0 mg, 200±0.9 mg, 200±0.8 mg, 200±0.7 mg, 200±0.6 mg, 200±0.5 mg, 200±0.4 mg, 200±0.3 mg, 200±0.2 mg, or 200±0.1 mg.

In some embodiments, Compound 67A is administered at a dosage of about 400±10 mg, 400±9 mg, 400±8 mg, 400±7 mg, 400±6 mg, 400±5 mg, 400±4.5 mg, 400±4.0 mg, 400±3.5 mg, 400±3.0 mg, 400±2.5 mg, 400±2.0 mg, 400±1.5 mg, 400±1.0 mg, 400±0.9 mg, 400±0.8 mg, 400±0.7 mg, 400±0.6 mg, 400±0.5 mg, 400±0.4 mg, 400±0.3 mg, 400±0.2 mg, or 400±0.1 mg.

In some embodiments, Compound 67A is administered at a dosage of about 500±10 mg, 500±9 mg, 500±8 mg, 500±7 mg, 500±6 mg, 500±5 mg, 500±4.5 mg, 500±4.0 mg, 500±3.5 mg, 500±3.0 mg, 500±2.5 mg, 500±2.0 mg, 500±1.5 mg, 500±1.0 mg, 500±0.9 mg, 500±0.8 mg, 500±0.7 mg, 500±0.6 mg, 500±0.5 mg, 500±0.4 mg, 500±0.3 mg, 500±0.2 mg, or 500±0.1 mg.

In some embodiments, Compound 67A is administered at a dosage of about 600±10 mg, 600±9 mg, 600±8 mg, 600±7 mg, 600±6 mg, 600±5 mg, 600±4.5 mg, 600±4.0 mg, 600±3.5 mg, 600±3.0 mg, 600±2.5 mg, 600±2.0 mg, 600±1.5 mg, 600±1.0 mg, 600±0.9 mg, 600±0.8 mg, 600±0.7 mg, 600±0.6 mg, 600±0.5 mg, 600±0.4 mg, 600±0.3 mg, 600±0.2 mg, or 600±0.1 mg.

In some embodiments, Compound 67A is administered at a dosage ranging from about 15 mg to about 650 mg, about 15 mg to about 600 mg, about 15 mg to about 550 mg, about 15 mg to about 500 mg, about 15 mg to about 450 mg, about 15 mg to about 400 mg, about 15 mg to about 350 mg, about 15 mg to about 300 mg, about 15 mg to about 250 mg, about 15 mg to about 200 mg, about 15 mg to about 150 mg, about 15 mg to about 140 mg, about 15 mg to about 130 mg, about 15 mg to about 120 mg, about 15 mg to about 100 mg, about 15 mg to about 90 mg, about 15 mg to about 80 mg, about 15 mg to about 70 mg, about 15 mg to about 60 mg, about 15 mg to about 50 mg, about 15 mg to about 45 mg, about 15 mg to about 40 mg, about 15 mg to about 35 mg, about 15 mg to about 30 mg, about 15 mg to about 25 mg, or about 15 mg to about 20 mg.

In some embodiments, Compound 67A is administered at a dosage ranging from about 20 mg to about 700 mg, about 25 mg to about 700 mg, about 30 mg to about 700 mg, about 35 mg to about 700 mg, about 40 mg to about 700 mg, about 45 mg to about 700 mg, about 50 mg to about 700 mg, about 60 mg to about 700 mg, about 70 mg to about 700 mg, about 80 mg to about 700 mg, about 90 mg to about 700 mg, about 100 mg to about 700 mg, about 110 mg to about 700 mg, about 120 mg to about 700 mg, about 130 mg to about 700 mg, about 140 mg to about 700 mg, about 150 mg to about 700 mg, about 200 mg to about 700 mg, about 250 mg to about 700 mg, about 300 mg to about 700 mg, about 350 mg to about 700 mg, about 400 mg to about 700 mg, about 450 mg to about 700 mg, about 500 mg to about 700 mg, about 550 mg to about 700 mg, about 600 mg to about 700 mg, or about 650 mg to about 700 mg.

In some embodiments, Compound 67A is administered at a dosage ranging from about 20 mg to about 600 mg, about 25 mg to about 600 mg, about 30 mg to about 600 mg, about 35 mg to about 600 mg, about 40 mg to about 600 mg, about 45 mg to about 600 mg, about 50 mg to about 600 mg, about 60 mg to about 600 mg, about 70 mg to about 600 mg, about 80 mg to about 600 mg, about 90 mg to about 600 mg, about 100 mg to about 600 mg, about 110 mg to about 600 mg, about 120 mg to about 600 mg, about 130 mg to about 600 mg, about 140 mg to about 600 mg, about 150 mg to about 600 mg, about 200 mg to about 600 mg, about 250 mg to about 600 mg, about 300 mg to about 600 mg, about 350 mg to about 600 mg, about 400 mg to about 600 mg, about 450 mg to about 600 mg, about 500 mg to about 600 mg, or about 550 mg to about 600 mg.

In some embodiments, Compound 67A is administered at a dosage ranging from about 20 mg to about 500 mg, about 25 mg to about 500 mg, about 30 mg to about 500 mg, about 35 mg to about 500 mg, about 40 mg to about 500 mg, about 45 mg to about 500 mg, about 50 mg to about 500 mg, about 60 mg to about 500 mg, about 70 mg to about 500 mg, about 80 mg to about 500 mg, about 90 mg to about 500 mg, about 100 mg to about 500 mg, about 110 mg to about 500 mg, about 120 mg to about 500 mg, about 130 mg to about 500 mg, about 140 mg to about 500 mg, about 150 mg to about 500 mg, about 200 mg to about 500 mg, about 250 mg to about 500 mg, about 300 mg to about 500 mg, about 350 mg to about 500 mg, about 400 mg to about 500 mg, or about 450 mg to about 500 mg.

In some embodiments, Compound 67A is administered at a dosage ranging from about 20 mg to about 400 mg, about 25 mg to about 400 mg, about 30 mg to about 400 mg, about 35 mg to about 400 mg, about 40 mg to about 400 mg, about 45 mg to about 400 mg, about 50 mg to about 400 mg, about 60 mg to about 400 mg, about 70 mg to about 400 mg, about 80 mg to about 400 mg, about 90 mg to about 400 mg, about 100 mg to about 400 mg, about 110 mg to about 400 mg, about 120 mg to about 400 mg, about 130 mg to about 400 mg, about 140 mg to about 400 mg, about 150 mg to about 400 mg, about 200 mg to about 400 mg, about 250 mg to about 400 mg, about 300 mg to about 400 mg, or about 350 mg to about 400 mg.

In some embodiments, Compound 67A is administered at a dosage ranging from about 20 mg to about 300 mg, about 25 mg to about 300 mg, about 30 mg to about 300 mg, about 35 mg to about 300 mg, about 40 mg to about 300 mg, about 45 mg to about 300 mg, about 50 mg to about 300 mg, about 60 mg to about 300 mg, about 70 mg to about 300 mg, about 80 mg to about 300 mg, about 90 mg to about 300 mg, about 100 mg to about 300 mg, about 110 mg to about 300 mg, about 120 mg to about 300 mg, about 130 mg to about 300 mg, about 140 mg to about 300 mg, about 150 mg to about 300 mg, about 200 mg to about 300 mg, or about 250 mg to about 300 mg.

In some embodiments, Compound 67A is administered at a dosage ranging from about 20 mg to about 200 mg, about 25 mg to about 200 mg, about 30 mg to about 200 mg, about 35 mg to about 200 mg, about 40 mg to about 200 mg, about 45 mg to about 200 mg, about 50 mg to about 200 mg, about 60 mg to about 200 mg, about 70 mg to about 200 mg, about 80 mg to about 200 mg, about 90 mg to about 200 mg, about 100 mg to about 200 mg, about 110 mg to about 200 mg, about 120 mg to about 200 mg, about 130 mg to about 200 mg, about 140 mg to about 200 mg, about 150 mg to about 200 mg.

In some embodiments, Compound 67A is administered at a dosage ranging from about 20 mg to about 150 mg, about 25 mg to about 150 mg, about 30 mg to about 150 mg, about 35 mg to about 150 mg, about 40 mg to about 150 mg, about 45 mg to about 150 mg, about 50 mg to about 150 mg, about 60 mg to about 150 mg, about 70 mg to about 150 mg, about 80 mg to about 150 mg, about 90 mg to about 150 mg, about 100 mg to about 150 mg, about 110 mg to about 150 mg, about 120 mg to about 150 mg, about 130 mg to about 150 mg, or about 140 mg to about 150 mg.

In some embodiments, Compound 67A is administered at a dosage ranging from about 20 mg to about 100 mg, about 25 mg to about 100 mg, about 30 mg to about 100 mg, about 35 mg to about 100 mg, about 40 mg to about 100 mg, about 45 mg to about 100 mg, about 50 mg to about 100 mg, about 60 mg to about 100 mg, about 70 mg to about 100 mg, about 80 mg to about 100 mg, or about 90 mg to about 100 mg.

In some embodiments, Compound 67A is administered at a dosage ranging from about 90 mg to about 700 mg, about 100 mg to about 200 mg, about 100 mg to about 300 mg, about 100 mg to about 400 mg, about 100 mg to about 500 mg, about 100 mg to about 600 mg, about 100 mg to about 700 mg, about 200 mg to about 300 mg, about 200 mg to about 400 mg, about 200 mg to about 500 mg, about 200 mg to about 600 mg, about 200 mg to about 700 mg, about 300 mg to about 400 mg, about 300 mg to about 500 mg, about 300 mg to about 600 mg, about 300 mg to about 700 mg, about 400 mg to about 500 mg, about 400 mg to about 600 mg, about 400 mg to about 700 mg, about 500 mg to about 600 mg, about 500 mg to about 700 mg, and about 600 mg to about 700 mg.

In some embodiments, Compound 67A is administered at a dosage ranging from about 15 mg to about 50 mg, about 15 mg to about 40 mg, about 15 mg to about 30 mg, about 20 mg to about 50 mg, about 20 mg to about 40 mg, about 20 mg to about 30 mg, about 30 mg to about 50 mg, and about 30 mg to about 40 mg.

In some embodiments, Compound 67A is administered at a dosage of about 15 mg/day.

In some embodiments, Compound 67A is administered at a dosage of about 20 mg/day.

In some embodiments, Compound 67A is administered at a dosage of about 25 mg/day.

In some embodiments, Compound 67A is administered at a dosage of about 30 mg/day.

In some embodiments, Compound 67A is administered at a dosage of about 35 mg/day.

In some embodiments, Compound 67A is administered at a dosage of about 40 mg/day.

In some embodiments, Compound 67A is administered at a dosage of about 45 mg/day.

In some embodiments, Compound 67A is administered at a dosage of about 50 mg/day.

In some embodiments, Compound 67A is administered at a dosage of about 55 mg/day.

In some embodiments, Compound 67A is administered at a dosage of about 60 mg/day.

In some embodiments, Compound 67A is administered at a dosage of about 65 mg/day.

In some embodiments, Compound 67A is administered at a dosage of about 70 mg/day.

In some embodiments, Compound 67A is administered at a dosage of about 75 mg/day.

In some embodiments, Compound 67A is administered at a dosage of about 80 mg/day.

In some embodiments, Compound 67A is administered at a dosage of about 85 mg/day.

In some embodiments, Compound 67A is administered at a dosage of about 90 mg/day.

In some embodiments, Compound 67A is administered at a dosage of about 95 mg/day.

In some embodiments, Compound 67A is administered at a dosage of about 100 mg/day.

In some embodiments, Compound 67A is administered at a dosage of about 110 mg/day.

In some embodiments, Compound 67A is administered at a dosage of about 120 mg/day.

In some embodiments, Compound 67A is administered at a dosage of about 130 mg/day.

In some embodiments, Compound 67A is administered at a dosage of about 140 mg/day.

In some embodiments, Compound 67A is administered at a dosage of about 150 mg/day.

In some embodiments, Compound 67A is administered at a dosage of about 160 mg/day.

In some embodiments, Compound 67A is administered at a dosage of about 170 mg/day.

In some embodiments, Compound 67A is administered at a dosage of about 180 mg/day.

In some embodiments, Compound 67A is administered at a dosage of about 190 mg/day.

In some embodiments, Compound 67A is administered at a dosage of about 200 mg/day.

In some embodiments, Compound 67A is administered at a dosage of about 220 mg/day.

In some embodiments, Compound 67A is administered at a dosage of about 240 mg/day.

In some embodiments, Compound 67A is administered at a dosage of about 260 mg/day.

In some embodiments, Compound 67A is administered at a dosage of about 280 mg/day.

In some embodiments, Compound 67A is administered at a dosage of about 300 mg/day.

In some embodiments, Compound 67A is administered at a dosage of about 320 mg/day.

In some embodiments, Compound 67A is administered at a dosage of about 340 mg/day.

In some embodiments, Compound 67A is administered at a dosage of about 360 mg/day.

In some embodiments, Compound 67A is administered at a dosage of about 380 mg/day.

In some embodiments, Compound 67A is administered at a dosage of about 400 mg/day.

In some embodiments, Compound 67A is administered at a dosage of about 420 mg/day.

In some embodiments, Compound 67A is administered at a dosage of about 440 mg/day.

In some embodiments, Compound 67A is administered at a dosage of about 460 mg/day.

In some embodiments, Compound 67A is administered at a dosage of about 480 mg/day.

In some embodiments, Compound 67A is administered at a dosage of about 500 mg/day.

In some embodiments, Compound 67A is administered at a dosage of about 520 mg/day.

In some embodiments, Compound 67A is administered at a dosage of about 540 mg/day.

In some embodiments, Compound 67A is administered at a dosage of about 560 mg/day.

In some embodiments, Compound 67A is administered at a dosage of about 580 mg/day.

In some embodiments, Compound 67A is administered at a dosage of about 600 mg/day.

In some embodiments, Compound 67A is administered at a dosage of about 620 mg/day.

In some embodiments, Compound 67A is administered at a dosage of about 640 mg/day.

In some embodiments, Compound 67A is administered at a dosage of about 660 mg/day.

In some embodiments, Compound 67A is administered at a dosage of about 680 mg/day.

In some embodiments, Compound 67A is administered at a dosage of about 700 mg/day.

In some embodiments, Compound 67A is administered at a dosage ranging from about 15 mg/day to about 1400 mg/day.

In some embodiments, Compound 67A is administered at a dosage ranging from about 15 mg/day to about 1200 mg/day.

In some embodiments, Compound 67A is administered at a dosage ranging from about 15 mg/day to about 1000 mg/day.

In some embodiments, Compound 67A is administered at a dosage ranging from about 15 mg/day to about 900 mg/day.

In some embodiments, Compound 67A is administered at a dosage ranging from about 15 mg/day to about 800 mg/day.

In some embodiments, Compound 67A is administered at a dosage ranging from about 15 mg/day to about 700 mg/day.

In some embodiments, Compound 67A is administered at a dosage ranging from about 15 mg/day to about 600 mg/day.

In some embodiments, Compound 67A is administered at a dosage ranging from about 15 mg/day to about 500 mg/day.

In some embodiments, Compound 67A is administered at a dosage ranging from about 15 mg/day to about 400 mg/day.

In some embodiments, Compound 67A is administered at a dosage ranging from about 15 mg/day to about 300 mg/day.

In some embodiments, Compound 67A is administered at a dosage ranging from about 15 mg/day to about 200 mg/day.

In some embodiments, Compound 67A is administered at a dosage ranging from about 15 mg/day to about 100 mg/day.

In some embodiments, Compound 67A is administered at a dosage ranging from about 15 mg/day to about 95 mg/day.

In some embodiments, Compound 67A is administered at a dosage ranging from about 15 mg/day to about 90 mg/day.

In some embodiments, Compound 67A is administered at a dosage ranging from about 15 mg/day to about 85 mg/day.

In some embodiments, Compound 67A is administered at a dosage ranging from about 15 mg/day to about 80 mg/day.

In some embodiments, Compound 67A is administered at a dosage ranging from about 15 mg/day to about 75 mg/day.

In some embodiments, Compound 67A is administered at a dosage ranging from about 15 mg/day to about 70 mg/day.

In some embodiments, Compound 67A is administered at a dosage ranging from about 15 mg/day to about 65 mg/day.

In some embodiments, Compound 67A is administered at a dosage ranging from about 15 mg/day to about 60 mg/day.

In some embodiments, Compound 67A is administered at a dosage ranging from about 15 mg/day to about 55 mg/day.

In some embodiments, Compound 67A is administered at a dosage ranging from about 15 mg/day to about 50 mg/day.

In some embodiments, Compound 67A is administered at a dosage ranging from about 15 mg/day to about 45 mg/day.

In some embodiments, Compound 67A is administered at a dosage ranging from about 15 mg/day to about 40 mg/day.

In some embodiments, Compound 67A is administered at a dosage ranging from about 15 mg/day to about 35 mg/day.

In some embodiments, Compound 67A is administered at a dosage ranging from about 15 mg/day to about 30 mg/day.

In some embodiments, Compound 67A is administered at a dosage ranging from about 15 mg/day to about 25 mg/day.

In some embodiments, Compound 67A is administered at a dosage ranging from about 15 mg/day to about 20 mg/day.

In some embodiments, Compound 67A is administered at a dosage ranging from about 20 mg/day to about 1400 mg/day.

In some embodiments, Compound 67A is administered at a dosage ranging from about 25 mg/day to about 1400 mg/day.

In some embodiments, Compound 67A is administered at a dosage ranging from about 30 mg/day to about 1400 mg/day.

In some embodiments, Compound 67A is administered at a dosage ranging from about 35 mg/day to about 1400 mg/day.

In some embodiments, Compound 67A is administered at a dosage ranging from about 40 mg/day to about 1400 mg/day.

In some embodiments, Compound 67A is administered at a dosage ranging from about 45 mg/day to about 1400 mg/day.

In some embodiments, Compound 67A is administered at a dosage ranging from about 50 mg/day to about 1400 mg/day.

In some embodiments, Compound 67A is administered at a dosage ranging from about 55 mg/day to about 1400 mg/day.

In some embodiments, Compound 67A is administered at a dosage ranging from about 60 mg/day to about 1400 mg/day.

In some embodiments, Compound 67A is administered at a dosage ranging from about 65 mg/day to about 1400 mg/day.

In some embodiments, Compound 67A is administered at a dosage ranging from about 70 mg/day to about 1400 mg/day.

In some embodiments, Compound 67A is administered at a dosage ranging from about 75 mg/day to about 1400 mg/day.

In some embodiments, Compound 67A is administered at a dosage ranging from about 80 mg/day to about 1400 mg/day.

In some embodiments, Compound 67A is administered at a dosage ranging from about 85 mg/day to about 1400 mg/day.

In some embodiments, Compound 67A is administered at a dosage ranging from about 90 mg/day to about 1400 mg/day.

In some embodiments, Compound 67A is administered at a dosage ranging from about 95 mg/day to about 1400 mg/day.

In some embodiments, Compound 67A is administered at a dosage ranging from about 100 mg/day to about 1400 mg/day.

In some embodiments, Compound 67A is administered at a dosage ranging from about 200 mg/day to about 1400 mg/day.

In some embodiments, Compound 67A is administered at a dosage ranging from about 300 mg/day to about 1400 mg/day.

In some embodiments, Compound 67A is administered at a dosage ranging from about 400 mg/day to about 1400 mg/day.

In some embodiments, Compound 67A is administered at a dosage ranging from about 500 mg/day to about 1400 mg/day.

In some embodiments, Compound 67A is administered at a dosage ranging from about 600 mg/day to about 1400 mg/day.

In some embodiments, Compound 67A is administered at a dosage ranging from about 700 mg/day to about 1400 mg/day.

In some embodiments, Compound 67A is administered at a dosage ranging from about 800 mg/day to about 1400 mg/day.

In some embodiments, Compound 67A is administered at a dosage ranging from about 900 mg/day to about 1400 mg/day.

In some embodiments, Compound 67A is administered at a dosage ranging from about 1000 mg/day to about 1400 mg/day.

In some embodiments, Compound 67A is administered at a dosage ranging from about 1100 mg/day to about 1400 mg/day.

In some embodiments, Compound 67A is administered at a dosage ranging from about 1200 mg/day to about 1400 mg/day.

In some embodiments, Compound 67A is administered at a dosage ranging from about 1300 mg/day to about 1400 mg/day.

In some embodiments, Compound 67A is administered at a dosage ranging from about 15 mg/day to about 1200 mg/day.

In some embodiments, Compound 67A is administered at a dosage ranging from about 20 mg/day to about 1200 mg/day.

In some embodiments, Compound 67A is administered at a dosage ranging from about 25 mg/day to about 1200 mg/day.

In some embodiments, Compound 67A is administered at a dosage ranging from about 30 mg/day to about 1200 mg/day.

In some embodiments, Compound 67A is administered at a dosage ranging from about 35 mg/day to about 1200 mg/day.

In some embodiments, Compound 67A is administered at a dosage ranging from about 40 mg/day to about 1200 mg/day.

In some embodiments, Compound 67A is administered at a dosage ranging from about 45 mg/day to about 1200 mg/day.

In some embodiments, Compound 67A is administered at a dosage ranging from about 50 mg/day to about 1200 mg/day.

In some embodiments, Compound 67A is administered at a dosage ranging from about 55 mg/day to about 1200 mg/day.

In some embodiments, Compound 67A is administered at a dosage ranging from about 60 mg/day to about 1200 mg/day.

In some embodiments, Compound 67A is administered at a dosage ranging from about 65 mg/day to about 1200 mg/day.

In some embodiments, Compound 67A is administered at a dosage ranging from about 70 mg/day to about 1200 mg/day.

In some embodiments, Compound 67A is administered at a dosage ranging from about 75 mg/day to about 1200 mg/day.

In some embodiments, Compound 67A is administered at a dosage ranging from about 80 mg/day to about 1200 mg/day.

In some embodiments, Compound 67A is administered at a dosage ranging from about 85 mg/day to about 1200 mg/day.

In some embodiments, Compound 67A is administered at a dosage ranging from about 90 mg/day to about 1200 mg/day.

In some embodiments, Compound 67A is administered at a dosage ranging from about 95 mg/day to about 1200 mg/day.

In some embodiments, Compound 67A is administered at a dosage ranging from about 100 mg/day to about 1200 mg/day.

In some embodiments, Compound 67A is administered at a dosage ranging from about 200 mg/day to about 1200 mg/day.

In some embodiments, Compound 67A is administered at a dosage ranging from about 300 mg/day to about 1200 mg/day.

In some embodiments, Compound 67A is administered at a dosage ranging from about 400 mg/day to about 1200 mg/day.

In some embodiments, Compound 67A is administered at a dosage ranging from about 500 mg/day to about 1200 mg/day.

In some embodiments, Compound 67A is administered at a dosage ranging from about 600 mg/day to about 1200 mg/day.

In some embodiments, Compound 67A is administered at a dosage ranging from about 700 mg/day to about 1200 mg/day.

In some embodiments, Compound 67A is administered at a dosage ranging from about 800 mg/day to about 1200 mg/day.

In some embodiments, Compound 67A is administered at a dosage ranging from about 900 mg/day to about 1200 mg/day.

In some embodiments, Compound 67A is administered at a dosage ranging from about 1000 mg/day to about 1200 mg/day.

In some embodiments, Compound 67A is administered at a dosage ranging from about 1100 mg/day to about 1200 mg/day.

In some embodiments, Compound 67A is administered once daily.

In some embodiments, Compound 67A is administered once daily at a dosage of about 700 mg.

In some embodiments, Compound 67A is administered once daily at a dosage of about 600 mg.

In some embodiments, Compound 67A is administered once daily at a dosage of about 500 mg.

In some embodiments, Compound 67A is administered once daily at a dosage of about 400 mg.

In some embodiments, Compound 67A is administered once daily at a dosage of about 300 mg.

In some embodiments, Compound 67A is administered once daily at a dosage of about 200 mg.

In some embodiments, Compound 67A is administered once daily at a dosage of about 130 mg.

In some embodiments, Compound 67A is administered once daily at a dosage of about 90 mg.

In some embodiments, Compound 67A is administered once daily at a dosage of about 45 mg.

In some embodiments, Compound 67A is administered once daily at a dosage of about 30 mg.

In some embodiments, Compound 67A is administered once daily at a dosage of about 20 mg.

In some embodiments, Compound 67A is administered once daily at a dosage of about 15 mg.

In some embodiments, Compound 67A is administered twice daily.

In some embodiments, Compound 67A is administered twice daily at a dosage of about 700 mg.

In some embodiments, Compound 67A is administered twice daily at a dosage of about 600 mg.

In some embodiments, Compound 67A is administered twice daily at a dosage of about 500 mg.

In some embodiments, Compound 67A is administered twice daily at a dosage of about 400 mg.

In some embodiments, Compound 67A is administered twice daily at a dosage of about 300 mg.

In some embodiments, Compound 67A is administered twice daily at a dosage of about 200 mg.

In some embodiments, Compound 67A is administered twice daily at a dosage of about 130 mg.

In some embodiments, Compound 67A is administered twice daily at a dosage of about 90 mg.

In some embodiments, Compound 67A is administered twice daily at a dosage of about 45 mg.

In some embodiments, Compound 67A is administered twice daily at a dosage of about 30 mg.

In some embodiments, Compound 67A is administered twice daily at a dosage of about 20 mg.

In some embodiments, Compound 67A is administered twice daily at a dosage of about 15 mg.

In some embodiments, Compound 67A is administered once daily, for example, at any of the dosage described herein.

In some embodiments, Compound 67A is administered once daily, for example, at any of the dosage described herein, for one day per week.

In some embodiments, Compound 67A is administered once daily, for example, at any of the dosage described herein, for two days per week.

In some embodiments, Compound 67A is administered once daily, for example, at any of the dosage described herein, for three days per week.

In some embodiments, Compound 67A is administered once daily, for example, at any of the dosage described herein, for four days per week.

In some embodiments, Compound 67A is administered once daily, for example, at any of the dosage described herein, for five days per week.

In some embodiments, Compound 67A is administered once daily, for example, at any of the dosage described herein, for six days per week.

In some embodiments, Compound 67A is administered once daily, for example, at any of the dosage described herein, for seven days per week.

In some embodiments, Compound 67A is administered once daily, for example, at any of the dosage described herein, for two weeks.

In some embodiments, Compound 67A is administered once daily, for example, at any of the dosage described herein, for three weeks.

In some embodiments, Compound 67A is administered once daily, for example, at any of the dosage described herein, for four weeks.

In some embodiments, Compound 67A is administered, for example, at any of the dosage described herein, once every other day.

In some embodiments, Compound 67A is administered, for example, at any of the dosage described herein, once every other day for four days.

In some embodiments, Compound 67A is administered, for example, at any of the dosage described herein, once every other day for 6 days.

In some embodiments, Compound 67A is administered, for example, at any of the dosage described herein, once every other day for two weeks.

In some embodiments, Compound 67A is administered, for example, at any of the dosage described herein, once every other day for three weeks.

In some embodiments, Compound 67A is administered, for example, at any of the dosage described herein, once every other day for four weeks.

In some embodiment, Compound 67A is administered, for example, at any of the dosage described herein, without a dosing holiday.

In some embodiment, Compound 67A is administered, for example, at any of the dosage described herein, followed by a dosing holiday.

In some embodiment, Compound 67A is administered, for example, at any of the dosage described herein, for two weeks followed by a dosing holiday.

In some embodiment, Compound 67A is administered, for example, at any of the dosage described herein, for three weeks followed by a dosing holiday.

In some embodiment, Compound 67A is administered, for example, at any of the dosage described herein, for four weeks followed by a dosing holiday.

In some embodiment, Compound 67A is administered, for example, at any of the dosage described herein, followed by a 7-day dosing holiday.

In some embodiments, Compound 67A is administered, for example, at any of the dosage described herein, followed by a 14-day dosing holiday.

In some embodiments, Compound 67A is administered, for example, at any of the dosage described herein, followed by a 21-day dosing holiday.

In some embodiments, Compound 67A is administered, for example, at any of the dosage described herein, followed by a 28-day dosing holiday.

In some embodiments, Compound 67A is administered, for example, at any of the dosage described herein, followed by a 35-day dosing holiday.

In some embodiments, Compound 67A is administered, for example, at any of the dosage described herein, followed by a 42-day dosing holiday.

In some embodiments, Compound 67A is administered, for example, at any of the dosage described herein, followed by a 49-day dosing holiday.

In some embodiments, Compound 67A is administered once a day continuously.

In some embodiments, Compound 67A is administered twice a day continuously.

In some embodiments, Compound 67A is administered orally.

Also included is Compound 67A, both in the pharmaceutically acceptable salt form and in the neutral form.

The term “pharmaceutically acceptable salt” refers to a pharmaceutical salt that is, within the scope of sound medical judgment, suitable for use in contact with the tissues of humans and lower animals without undue toxicity, irritation, and allergic response, and is commensurate with a reasonable benefit/risk ratio. Pharmaceutically-acceptable salts are well known in the art. For example, S. M. Berge et al. describes pharmacologically acceptable salts in J. Pharm. Sci., 1977, 66, 1-19.

Included in the present teachings are pharmaceutically acceptable salts of Compound 67A. Compounds having basic groups can form pharmaceutically acceptable salts with pharmaceutically acceptable acid(s). Suitable pharmaceutically acceptable acid addition salts of the compounds described herein include salts of inorganic acids (such as hydrochloric acid, hydrobromic, phosphoric, metaphosphoric, nitric, and sulfuric acids) and of organic acids (such as acetic acid, benzenesulfonic, benzoic, ethanesulfonic, methanesulfonic, succinic, and trifluoroacetic acid acids). Compounds of the present teachings with acidic groups such as carboxylic acids can form pharmaceutically acceptable salts with pharmaceutically acceptable base(s). Suitable pharmaceutically acceptable basic salts include ammonium salts, alkali metal salts (such as sodium and potassium salts) and alkaline earth metal salts (such as magnesium and calcium salts).

Unless explicitly indicated otherwise, the terms “approximately” and “about” are synonymous. In one embodiment, “approximately” and “about” refer to the recited amount, value, or duration ±5%, ±4.5%, ±4%, ±3.5%, ±3%, ±2.5%, ±2%, ±1.75%, ±1.5%, ±1.25%, ±1%, ±0.9%, ±0.8%, ±0.7%, ±0.6%, ±0.5%±0.4%, ±0.3%, ±0.2%, ±0.1%, ±0.09%, ±0.08%, ±0.07%, ±0.06%, ±0.05%, ±0.04%, ±0.03%, ±0.02%, or ±0.01%. In another embodiment, “approximately” and “about” refer to the listed amount, value, or duration ±2.5%, ±2%, ±1.75%, ±1.5%, ±1.25%, ±1%, ±0.9%, ±0.8%, ±0.7%, ±0.6%, ±0.5%. In yet another embodiment, “approximately” and “about” refer to the listed amount, value, or duration ±1%. In yet another embodiment, “approximately” and “about” refer to the listed amount, value, or duration ±0.5%. In yet another embodiment, “approximately” and “about” refer to the listed amount, value, or duration ±0.1%.

### Definitions

Compounds having one or more chiral centers can exist in various stereoisomeric forms. Stereoisomers are compounds that differ only in their spatial arrangement. Stereoisomers include all diastereomeric, enantiomeric, and epimeric forms as well as racemates and mixtures thereof.

The term “geometric isomer” refers to cyclic compounds having at least two substituents, wherein the two substituents are both on the same side of the ring (cis) or wherein the substituents are each on opposite sides of the ring (trans). When a disclosed compound is named or depicted by structure without indicating stereochemistry, it is understood that the name or the structure encompasses one or more of the possible stereoisomers, or geometric isomers, or a mixture of the encompassed stereoisomers or geometric isomers.

When a geometric isomer is depicted by name or structure, it is to be understood that the named or depicted isomer exists to a greater degree than another isomer, that is that the geometric isomeric purity of the named or depicted geometric isomer is greater than 50%, such as at least 60%, 70%, 80%, 90%, 99%, or 99.9% pure by weight. Geometric isomeric purity is determined by dividing the weight of the named or depicted geometric isomer in the mixture by the total weight of all of the geometric isomers in the mixture.

Racemic mixture means 50% of one enantiomer and 50% of is corresponding enantiomer. When a compound with one chiral center is named or depicted without indicating the stereochemistry of the chiral center, it is understood that the name or structure encompasses both possible enantiomeric forms (e.g., both enantiomerically-pure, enantiomerically-enriched or racemic) of the compound. When a compound with two or more chiral centers is named or depicted without indicating the stereochemistry of the chiral centers, it is understood that the name or structure encompasses all possible diastereomeric forms (e.g., diastereomerically pure, diastereomerically enriched and equimolar mixtures of one or more diastereomers (e.g., racemic mixtures) of the compound.

Enantiomeric and diastereomeric mixtures can be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and diastereomers also can be obtained from diastereomerically- or enantiomerically-pure intermediates, reagents, and catalysts by well-known asymmetric synthetic methods.

When a compound is designated by a name or structure that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers is included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

### Pharmaceutical Compositions

The compounds disclosed therein are RAD51 inhibitors. The pharmaceutical composition of the present disclosure comprises Compound 67A, or a pharmaceutically acceptable salt thereof, and a pharmaceutically acceptable carrier or diluent.

“Pharmaceutically acceptable carrier” and “pharmaceutically acceptable diluent” refer to a substance that aids the formulation and/or administration of an active agent to and/or absorption by a subject and can be included in the compositions of the present disclosure without causing a significant adverse toxicological effect on the subject. Non-limiting examples of pharmaceutically acceptable carriers and/or diluents include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds.

The pharmaceutical compositions of the present teachings optionally include one or more pharmaceutically acceptable carriers and/or diluents therefor, such as lactose, starch, cellulose and dextrose. Other excipients, such as flavoring agents; sweeteners; and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003—20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

The term “dosage holiday”, also referred to as “drug holiday,” refers to a period of time wherein the subject is not administered or administered at a lower dosage the therapeutic (i.e., RAD51 inhibitor). The timing of a dosage holiday depends on the timing of the regular dosing regimen and the purpose for taking the dosage holiday (e.g., to regain drug sensitivity and/or to reduce unwanted side effects of continuous, long-term administration). In some embodiments, the dosage holiday may be a reduction in the dosage of the drug (e.g., to below the therapeutically effective amount for a certain interval of time). In other embodiments, administration of the dosage is stopped for a certain interval of time before administration is started again at the same or different dosing regimen (e.g., at a lower or higher dose and/or frequency of administration). A dosage holiday of the disclosure may thus be selected from a wide range of time-periods and dosage regimens.

### Additional Aspects of Methods of Treatment

In some embodiments, the subject can be a subject determined to have an increased level of DNA damage occurring in one or more cell types relative to a reference level. As used herein, “DNA damage” refers to breaks, nicks, and mutations of the DNA present in a cell. In some embodiments, the DNA damage can comprise one or more of single-strand breaks (e.g., “nicks”), double strand breaks (DSBs), and mutations. In some embodiments, the DNA damage can be one or more DSBs. As used herein, “mutation” refers to a change or difference in the genetic material of a cell as compared to a reference wildtype cell, e.g. a deletion, an insertion, a SNP, a gene rearrangement, and/or the introduction of an exogenous gene or sequence.

In some embodiments, the subject can be determined to have an increased level of DNA damage if the subject is determined to have an increased level and/or activity of a DNA damage process or DNA editing enzyme. As used herein, “DNA damage process” refers to any activity or process in a cell which causes one or more types of DNA damage to occur.

In some embodiments, an increased level of DNA damage can be an increased level of mutations, e.g., by determining the overall mutation status in all or a portion of the genome of a cell. An overall mutation status at least 2% greater, e.g. 2% greater or more, 3% greater or more, 5% greater or more, 10% greater or more, or 20% greater or more than the overall mutation status in a reference cell can be indicative of an increased, elevated, and/or significant level of a DNA editing enzyme activity. In some embodiments, the level of hyper mutations can be determined. In some embodiments, the overall mutation status in the whole genome or a portion thereof can be determined using FISH, whole genome sequencing, high throughput sequencing, exome sequencing, hybridization, and/or PCR. In some embodiments the activity of a DNA editing enzyme can be measured by determining the level of hypermutations in the specific target genes including, but not limited to IGH, BCL6, MYC, BCL11A, CD93, PIM1 and/or PAX5. In some embodiments the DNA editing enzyme is AID. In some embodiments, a level of mutation in specific target genes including IGH, BCL6, MYC, BCL1 1A, CD93, PIM1 and/or PAX5 which is at least 2% greater, e.g. 2% greater or more, 3% greater or more, 5% greater or more, 10% greater or more, or 20% greater or more than the level of mutation in IGH, BCL6, MYC, BCL1 1A, CD93, PIM1 and/or PAX5 in a reference cell can be indicative of an increased, elevated, and/or significant level of AID activity.

In some embodiments, an increased level of DNA damage can be an increased level of double strand breaks (DSBs). The level of DSBs can be determined, by way of non-limiting example, by karyotyping, by γ-H2AX foci formation, and/or by using FISH analysis to detect DNA double strand breaks, e.g. DNA breakage detection fish (DBD-FISH) (Volpi and Bridger, BioTechniques, Vol. 45, No. 4, October 2008, pp. 385-409).

In some embodiments, an increased level of DNA damage can be an increased level of single strand breaks. The level of single-strand breaks in DNA can be determined, by way of non-limiting example, by COMET assays, FISH, or the use of single-strand break-specific probes. Detection of DNA breaks, both single and double-stranded is known in the art and described further, at, e.g., Kumari et al. EXCLI Journal 2009 7:44-62 and Motalleb et al. Research Journal of Applied Sciences, Engineering and Technology. 2012 4: 1888-1894; each of which is incorporated by reference herein in its entirety.

In some embodiments, an increased level of activity of a DNA damage process can comprise an increased level and/or activity of a DNA editing enzyme. In some embodiments, the technology described herein is directed to treating cells having an active DNA editing enzyme with a compound of the present disclosure. In some embodiments, the technology described herein is directed to treating cells having an increased level and/or activity of a DNA editing enzyme with a compound of the present disclosure. As used herein, “DNA editing enzyme” refers to an enzyme which normally catalyzes the mutation, exchange or excision of DNA segments, particularly enzymes which can generate or promote the generation of point mutations, DNA single strand breaks, DNA double-strand breaks or protein-DNA adducts. A DNA editing enzyme, as referred to herein, is not necessarily site-specific in its action. Similarly, it is not necessarily cell specific. In some embodiments, the cell is a B cell expressing a detectable amount of such an enzyme.

Non-limiting examples of DNA editing enzymes include, but are not limited to Recombination Activating Gene 1 (RAG1; NCBI Gene ID: 5896), Recombination Activating Gene 1 (RAG2; NCBI Gene ID: 5897), Sporulation-specific protein 11 (SPO1 1; NCBI Gene ID: 23626), APOBEC family members a Type 1 Topoisomerase; a Type 2 Topoisomerase; and/or AID. In some embodiments, the DNA editing enzyme can be AID.

In some embodiments, the DNA editing enzyme can be a member of the APOBEC (apolipoprotein B mRNA editing enzyme, catalytic polypeptide-like) family. As used herein “APOBEC family” refers to a family of cytidine deaminase enzymes having an N-terminal zinc-dependent cytidine deaminase catalytic domain comprising and a C-terminal pseudocatalytic domain. Non-limiting examples of APOBEC family members include AID, APOBEC 1 (e.g., NCBI Gene ID: 339), APOBEC2 (e.g., NCBI Gene ID: 10930), APOBEC3A (e.g., NCBI Gene ID: 200315), APOBEC3B (e.g., NCBI Gene ID: 9582), APOBEC3C (e.g., NCBI Gene ID: 27350), APOBEC3D (e.g., NCBI Gene ID: 140564), APOBEC3E (e.g., NCBI Gene ID: 140564), APOBEC3F (e.g., NCBI Gene ID:200316), APOBEC3G (e.g., NCBI Gene ID: 60489), APOBEC3H (e.g., NCBI Gene ID: 164668), and APOBEC4 (e.g., NCBI Gene ID: 403314).

In some embodiments, the DNA editing enzyme can be a Type 1 topoisomerase. In some embodiments, the DNA editing enzyme can be a Type 2 topoisomerase. Topoisomerases generate breaks in DNA to help uncoil or relax the strand. Type II topoisomerases hydrolyze ATP to generate DSB cuts, while Type I topoisomerases generate single-stranded breaks. Non-limiting examples of Type II topoisomerases can include topoisomerase II (e.g., NCBI Gene ID: 7153 and 7155). Non-limiting examples of Type I topoisomerases can include topoisomerase I (e.g., NCBI Gene ID: 7150).

Embodiments of the technology described herein are based on the discovery that the compounds described herein can inhibit DNA repair mechanisms, e.g., homologous repair. Activation-induced cytidine deaminase (AID, or AICDA, also known as ARP2, CDA2 or HIGM2), a DNA-editing enzyme that is a member of the apolipoprotein B mRNA editing enzymes, catalytic polypeptide-like (APOBEC), will cause widespread genomic breaks and cell death in cells with diminished homologous recombination ability (e.g. cells with diminished DNA double strand break repair abilities). Accordingly, provided herein is a method of causing cell death comprising detecting increased expression of a DNA-editing enzyme (e.g. AID) in a cell and thereafter contacting the cell with a compound of the present disclosure; thereby resulting in cell death. Accordingly, provided herein is a method of causing cell death comprising increasing expression of a DNA-editing enzyme (e.g. AID) in a cell and thereafter contacting the cell with a compound of the present disclosure; thereby resulting in cell death. Accordingly, provided herein is a method of causing cell death comprising administering to a cell a therapeutically effective amount of a DNA editing enzyme (e.g. AID) and thereafter contacting the cell with a compound of the present disclosure; thereby resulting in cell death.

AID, encoded by the AICDA gene (NCBI Gene ID: 57379), is required for proper B-cell function and is most prominently expressed in centroblast B-cells. The protein is involved in somatic hypermutation, gene conversion, and class-switch recombination of immunoglobulin genes. AID is normally expressed almost exclusively in antigen-activated germinal center B-cells, where it initiates immunoglobulin isotype class switching (Manis et al. 2002, Trends Immunol, 23, 31-39; Chaudhuri and Alt, Nat Rev Immunol, 2004, 4, 541-552; Longerich et al., Curr Opin Immunol, 2006, 18, 164-174; Chaudhuri et al., Adv Immunol 2007, 94, 157-214). AID is required for somatic hypermutation and immunoglobulin class switching in activated B cells. AID expression is regulated by CD40 ligand, B-cell receptor, IL4R, or Toll-like receptor stimulation (Crouch et al., J Exp Med 2007 204: 1145-1156; Muramatsu et al., J Biol Chem 1999 274: 18470-6). After activation, AID is transiently upregulated, induces point mutations or DNA double strand breaks in a sequence nonspecific manner within immunoglobulin genes, and is then downregulated (Longerich et al., Curr Opin Immunol, 2006, 18, 164-176; Chaudhuri et al., Adv Immunol 2007, 94, 157-214). Overall, AID is active in only a tiny population of normal cells (antigen-activated B-cells) at any given time. The genomic rearrangements and mutations controlled by AID lead to the development of antigen-recognition diversity, receptor editing and lymphoid effector function required for functional adaptive immunity (Mills, et al. Immunol Rev 2003 194:77-95). Recently it has been reported that AID has off-target point mutation activities (Liu, M. et al., Nature 2008, 451, 841-845; Liu and Schatz, Trends Immunol. 2009, 30, 173-181; Perez-Duran et al., Carcinogenesis. 2007, 28(12):2427-33). Robbiani et al. has reported off-target activities of AID in B-cells, especially c-myc/IgH translocations (Robbiani et al., Mol Cell 2009, 36(4):631-41). AID expression accelerates the rate of tumor development in Bcl6 transgenic mice (Pasqualucci et al., 2008, Nat. Genet. 40, 108-112). However, deregulated AID does not necessarily cause malignancy or translocation-associated cancer on its own in B cells (Muto et al., 2006, Proc. Natl. Acad. Sci. USA 103, 2752-2757; Okazaki et al., 2003, J. Exp. Med. 197, 1173-1181; Shen et al., 2008, Mol. Immunol. 45, 1883-1892). In addition, despite its obligate role in c-myc/IgH translocation, AID is not required for the development of plasmacytosis or plasmacytoma in IL-6 transgenic or pristane-treated mice, respectively (Kovalchuk et al., 2007, J. Exp. Med. 204, 2989-3001; Ramiro et al., 2004, J. Exp. Med. 200, 1103-1110). However, most human B cell lymphoma-associated translocations do not involve c-myc, and many do not involve Ig genes (Kuppers, 2005, Oncogene 20, 5580-5594).

Overexpression of AID has been reported in chronic lymphocytic leukemia (CLL) (Hancer et al. Leuk Lymphoma. 2011 January; 52(1):79-84; Heintel et al., Leukemia. 2004 April; 18(4):756-62). Further, AID expression has been shown to be correlated with blast crisis B lineage leukemia and therapy resistance in myeloid leukemia and to be associated with generally poor prognosis in chronic B lymphocytic leukemia (Mao et al., Br J Dermatol 2001, 145: 117-122; Chaudhuri et al., Nature 2004, 430:992-8). Further expression of AID in tumor cells from a variety of cancers has been reported including but not limited to lung, breast, gastric, colon, intestinal, liver cancer and choriangiocarcinoma (Greeve et al., Blood 2003, 1010, 3574-3580; Feldhahn et al., J Exp Med 2007, 204, 1157-1166; Kotani et al., PNAS USA 2007, 104, 1616-1620; Engels et al., 2008, Appl Immunohistochem Mol Morphol 16, 521-529; Klemm et al., 2009, Cancer Cell 6, 232-245; Palacios et al., 2010, Blood 115(22), 4488-4496; Leuenberger et al., 2009, Mod Pathol 32, 177-186; Gruber et al., 2010, Cancer Res 70, 7411-7420; inflammatory cancer (Marusawa 2008, Int J Biochem Cell Biol. 40, 399-402); follicular lymphoma (Hardianti et al., 2004, Leukemia 18, 826-831; Shikata et al., 2012, Cancer Sci. 103(3):415-21); thyroid cancer (Qiu et al. 2012, Mod Pathol 25(l),36-45); breast cancer (Borchert et al. 2011, BMC Cancer 11:347); Marusawa, et al., 2011, Adv Immunol 111: 109-41; Zhang et al. 2012, Hum Pathol 43(3):423-34; Komori et al., 2008, Hepatology 47(3):888-896; Hockley 2010, Leukemia 24(5): 1084-6; adult T-cell leukemia (Nakamura et al., 2011, Br J Dermatol. 165(2):437-9). All of the references in the foregoing paragraph are incorporated by reference herein in their entireties.

Elevated levels of AID have been reported in arthritis (Xu et al. Scand. J. Immunol. 2009, 296, 2033-6) and in the MRL/Fas(lpr/lpr) mouse lupus model (White et al. 2011, Autoimmunity 44(8), 585-98). All of the references in the foregoing paragraph are incorporated by reference herein in their entireties.

When DSB repair is inhibited, the extent of the DSBs generated by AID is much higher than previously suspected and the extent of genomic damage is so severe as to result in cell death. Accordingly, in one embodiment of the technology described herein, there is provided a method of treatment comprising; (a) selecting a subject having cells that express elevated levels of activation-induced cytidine deaminase (AID); and (b) administering a therapeutically effective amount of an inhibitor of double strand break repair (e.g. a compound of the present disclosure) to the subject; wherein an elevated level of AID is a level of AID that is higher than the level of AID in cells of the same type from a healthy individual. In some embodiments, the cells expressing elevated levels of AID are B cells. In some embodiments, the B cell expressing elevated levels of AID is a cancerous B cells or a B cell associated with autoimmune disease. In some embodiments, the subject can be a human subject.

Methods provided herein treat cancers and/or autoimmune disorders by inhibiting DNA double strand break repair. Methods provided herein treat pancreatic cancer by inhibiting DNA double strand break repair. This inhibition proves lethal to cells expressing AID, as AID generates widespread genomic breaks, and the treatment with a double strand break repair inhibitor prevents the repair of these lesions which are being generated by the cell itself. This results in cell death in the subject which is specific to the cells expressing AID, e.g. cancerous B cells and/or autoimmune cells. Accordingly, as described herein, in one embodiment there is a provided a treatment paradigm that selectively induces self-destruction of certain diseased cells, while reducing the unintended side effects in healthy tissues.

In some embodiments, an increased level and/or activity of a DNA editing enzyme can be an increased level of DNA editing enzyme mRNA. mRNA levels can be assessed using, e.g., biochemical and molecular biology techniques such as Northern blotting or other hybridization assays, nuclease protection assay, reverse transcription (quantitative RT-PCR) techniques, RNA-Seq, high throughput sequencing and the like. Such assays are well known to those in the art. In one embodiment, nuclear “run-on” (or “run-off) transcription assays are used (see e.g. Methods in Molecular Biology, Volume: 49, Sep. 27, 1995, Page Range: 229-238). Arrays can also be used; arrays, and methods of analyzing mRNA using such arrays have been described previously, e.g. in EP0834575, EP0834576, WO96/31622, U.S. Pat. No. 5,837,832 or WO98/30883. WO97/10365 provides methods for monitoring of expression levels of a multiplicity of genes using high density oligonucleotide arrays.

In some embodiments, a subject can be determined to have an increased level of DNA damage occurring in one or more cell types relative to a reference level if the subject has been exposed to an agent that is known to cause such DNA damage. Non-limiting examples of such agents can include a viral infection with a DNA integrating virus (e.g. adeno-associated virus, retrovirus, human T-lymphotropic virus, HIV-1, oncovirus, hepatitis virus, hepatitis B virus); DNA damaging chemicals (e.g. acetaldehyde, polycyclic aromatic hydrocarbons, benzenes, nitrosamines, tobacco smoke, aflatoxin, and the like); DNA damaging chemotherapeutic agents (e.g. bleomycin, mitomycin, nitrogen mustards (e.g. mechlorethamine, cyclophosphamide, melphalan, chlorambucil, ifosfamide and busulfan), nitrosoureas (e.g., N-Nitroso-N-methylurea (MNU), carmustine (BCNU), lomustine (CCNU) and semustine (MeCCNU), fotemustine and streptozotocin), tetrazines (e.g., dacarbazine, mitozolomide and temozolomide), aziridines (e.g., thiotepa, mytomycin and diaziquone (AZQ)), cisplatins (e.g., cisplatin, carboplatin and oxaliplatin) procarbazine and hexamethylmelamine); and ionizing or ultraviolet radiation. Exposure to such agents can be the result of an accident, infection and/or environmental exposure or the result of a therapeutic administration of such agents.

In some embodiments, the increased level of DNA damage can be occurring in a cell type affected by the cancer, autoimmune disease, and/or neurodegenerative disease. In some embodiments, the subject is determined to have an increased level of DNA damage occurring in a cell selected from the group consisting of: a cancer cell (e.g., pancreatic cancer cell); an immune system cell; or a nervous system cell.

In some embodiments, the DNA editing enzyme can be AID. In some embodiments, the level of AID can be the level of AID in a blood cell. In some embodiments, the level of AID can be the level of AID in a B cell.

In some embodiments, an increased level of AID can be a detectable level of AID, e.g., as described below herein.

In some embodiments, the subject can be a human subject.

Methods provided herein treat cancers and/or autoimmune disorders by inhibiting DNA double strand break repair. Methods provided herein treat a pancreatic cancer by inhibiting DNA double strand break repair. This inhibition proves lethal to cells expressing AID, as AID generates widespread genomic breaks, and the treatment with a double strand break repair inhibitor prevents the repair of these lesions which are being generated by the cell itself. This results in cell death in the subject which is specific to the cells expressing AID, e.g. cancerous B cells and/or autoimmune cells. Accordingly, as described herein, in one embodiment there is a provided a treatment paradigm that selectively induces self-destruction of certain diseased cells, while reducing the unintended side effects in healthy tissues.

Methods of detecting cancers in patients with increased levels of DNA damage or increased levels of DNA editing enzymes are disclosed in WO2016/094897, incorporated herein by reference.

In some embodiments, the cancer to be treated is a type with high expression of a DNA editing enzyme. In some embodiments, the cancer to be treated is a B-cell neoplasm.

Another embodiment is a method of treating a cancer by administering to the subject an effective amount of Compound 67A, or a pharmaceutically acceptable salt thereof, or the corresponding pharmaceutical composition. In one aspect, the cancer is selected from the group consisting of lymphoma, leukemia, and a plasma cell neoplasm. In some embodiments, the cancer is a carcinoma or a sarcoma.

In some embodiments, the cancer is B-cell non-Hodgkin's lymphoma, chronic lymphocytic leukemia, multiple myeloma, breast cancer, head and neck cancer, soft tissue sarcoma, ovarian cancer, pancreatic cancer, follicular lymphoma, or mantle cell lymphoma.

In some embodiments, the cancer to be treated is pancreatic cancer.

In some embodiments, the pancreatic cancer is exocrine pancreatic cancer. In some embodiments, the exocrine pancreatic cancer is adenocarcinoma. In some embodiments, the exocrine pancreatic cancer is squamous cell carcinoma. In some embodiments, the exocrine pancreatic cancer is adenosquamous carcinoma. In some embodiments, the exocrine pancreatic cancer is colloid carcinoma.

In some embodiments, the pancreatic cancer is neuroendocrine pancreatic cancer. In some embodiments, the neuroendocrine pancreatic cancer is a nonfunctioning neuroendocrine tumor. In some embodiments, the neuroendocrine pancreatic cancer is gastrinoma. In some embodiments, the neuroendocrine pancreatic cancer is insulinoma. In some embodiments, the neuroendocrine pancreatic cancer is glucagonoma.

In some embodiments, the neuroendocrine pancreatic cancer is a VlPoma (i.e., an islet cell tumor which affects the vasoactive intestinal peptides). In some embodiments, the neuroendocrine pancreatic cancer is a somatostatinoma (i.e., an islet cell tumor which affects the somatostatin).

In some embodiments, the pancreatic cancer is benign precancerous lesions.

In some embodiments, the cancer to be treated is a lymphoma. Lymphomas which can be treated by the disclosed methods include Non-Hodgkin's lymphoma; Burkitt's lymphoma; small lymphocytic lymphoma; lymphoplasmacytic lymphoma; MALT lymphoma; follicular lymphoma; diffuse large B-cell lymphoma; and T-cell lymphoma.

Lymphoma is a malignancy in the lymphatic cells of the immune system (e.g. B cells, T cells, or natural killer (NK) cells). Lymphomas often originate in the lymph nodes and present as solid tumors. They can metastasize to other organs such as the brain, bone, or skin. Extranodal sites are often located in the abdomen. Lymphomas are closely related to the lymphoid leukemia and in some cases a particular form of cancer is categorized as both a lymphoma and a leukemia.

Leukemias, which can be treated by the disclosed methods, include acute lymphoblastic leukemia (ALL); Burkitt's leukemia; B-cell leukemia; B-cell acute lymphoblastic leukemia; chronic lymphocytic leukemia (CLL); acute myelogenous leukemia (AML); chronic myelogenous leukemia (CIVIL); and T-cell acute lymphoblastic leukemia (T-ALL).

In some embodiments, the cancer to be treated is B-cell neoplasms, B-cell leukemia, B-cell acute lymphoblastic leukemia, chronic lymphocytic leukemia, chronic myelogenous leukemia, Burkitt's leukemia, acute myelogenous leukemia and/or T-ALL. The maturation of B cells most typically ceases or substantially decreases when the foreign antigen has been neutralized. Occasionally, however, proliferation of a particular B cell will continue unabated; such proliferation can result in a cancer referred to as “B-cell lymphoma” or a “B-cell leukemia.” In some embodiments the cancer to be treated is chronic lymphocytic leukemia (CLL) or chronic myelogenous leukemia (CML).

In some embodiments the cancer to be treated is a plasma cell neoplasm. Examples for plasma cell neoplasms include multiple myeloma; plasma cell myeloma; plasma cell leukemia and plasmacytoma.

Carcinomas which can be treated by the disclosed methods include colon cancer; liver cancer; gastric cancer; intestinal cancer; esophageal cancer; breast cancer; ovarian cancer; head and neck cancer; lung cancer; and thyroid cancer.

In some embodiments, the breast cancer is triple negative breast cancer.

Sarcomas which can be treated by the disclosed methods include soft tissue sarcoma and bone sarcoma.

Any cancer characterized by high levels of DNA damage and/or DNA editing enzyme expression can be treated with a compound as described herein, e.g. a compound of the present disclosure. For example, sarcomas, epithelial cell cancer (carcinomas), colon cancer, gastric cancer, intestinal cancer, liver cancer, hepatocellular cancer, breast cancer, thyroid cancer, esophageal cancer, lung cancer, brain cancer, head and neck cancer, melanoma, renal cancer, prostate cancer, hemangioma, rhabdomyosarcoma, chondrosarcoma, osteosarcoma, fibrosarcoma and cholangiocarcinoma may be characterized by high levels of a DNA editing enzyme expression, e.g. AID. In some embodiments the cancer to be treated is colon cancer, liver cancer, gastric cancer, intestinal cancer, breast cancer, lung cancer, thyroid cancer and/or cholangiocarcinoma.

In some embodiments, cancers that can be treated by the disclosed methods include, but are not limited to, cancer of the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may be of the following histological type, though it is not limited to these: neoplasm, malignant; carcinoma; carcinoma, undifferentiated; giant and spindle cell carcinoma; sarcomas; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer is relapsed or refractory.

In another embodiment for the disclosed method, the cancer is characterized by mutations in the mutS homologues (e.g., MSH2, MSH3, and MSH6), mutL homologues (e.g. MLH1), or mismatch repair endonuclease PMS2. Mutations are changes in the genetic code. They include point mutations and frameshift mutations. In a point mutation, one nucleotide is swapped out for another. Therefore, the mutation occurs at a single point or location within the DNA strand. Frameshift mutations are due to either insertions or deletions of nucleotides. This causes the entire DNA strand to elongate or to shrink in size. Thus, frameshift mutations may alter all of the codons that occur after the deletion or insertion. The mutations referred to herein include, but are not limited to, insertions, deletions, duplications, inversions, or other recognized point mutations. It has now been found that RAD51 inhibitors are particularly effective in treating cancers with mutations in MSH (e.g. MSH6), MLH, or PMS2.

MutS Homolog 2 (MSH2) is a protein that in humans is encoded by the MSH2 gene, which is located on chromosome 2. MSH2 is a tumor suppressor gene and, for example, a caretaker gene that codes for a DNA mismatch repair (MMR) protein, MSH2, which forms a heterodimer with MSH6 to make the human MutSα mismatch repair complex. It also dimerizes with MSH3 to form the MutSβ DNA repair complex. MSH2 is involved in many different forms of DNA repair, including transcription-coupled repair, homologous recombination, and base excision repair. Examples of the mutations in MSH2 include, but are not limited to, g.47630253_47630254del, g.47702411_47702421del, g.47709913_47709915inv, g.47635629_47635634del, g.47637227_47637236dup, g.47639550_47639561del, g.(?_47630206)_(47710367_?)del, g.(?_47630206)_(47643569_47656880)del, g.47630263_47643568del, g.(?_47630206)_(47657081_47672686)del, g.47630263_47657080del, g.(?_47630206)_(47672797_47690169)del, g.47630263_47672796del, g.(?_47630206)_(47672797_47690169)del, g.(?_47630206)_(47693948_47698103)del, g.47630263_47693947del, g.(?_47630206)_(47698202_47702163)del, g.(?_47630206)_(47630542_47635539)del, g.(?_47630206)_(47708011_47709917)del, g.(?_47630206)_(47635695_47637232)del, g.(?_47630206)_(47635695_47637232)del, g.(?_47630206)_(47637512_47639552)del, g.(?_47630206)_(47639700_47641407)del, g.(?_47630206)_(47641558_47643434)del, g.47618487_47650860delins(155), g.47628578_47638433del, g.47595033_47662777del, g.47583175_47667707del, g.47625602_47636880del, g.47554933_47699909del, g.47629508_47649552del, g.47629375_47651274del, g.(?_47630206)_(47630542_47635539)del, g.(?_47630206)_(47635695_47637232)del, g.47643509_47643510del, g.47643529_47643530dup, g.47656746_47657199dup, g.47656661_47663325del, g.(47643569_47656880)_(47710367_?)del, g.(47643569_47656880)_(47710367_?)del, g.47656881_47657080del, g.(47643569_47656880)_(47657081_47672686)del, g.(47643569_47656880)_(47657081_47672686)del, g.(47643569_47656880)_(47657081_47672686)del, g.(47643569_47656880)_(47657081_47672686)dup, g.(47643569_47656880)_(47657081_47672686)dup, g.(47643569_47656880)_(47672797_47690169)del, g.(47643569_47656880)_(47693948_47698103)del, g.47656881_47693947del, g.(47643569_47656880)_(47702410_47703505)del, g.47656881_47656882ins(173), g.47656901_47656902insA, g.47656903del, g.47656912del, g.47630440del, g.47656923del, g.47656931_47656932dup, g.47656943del, g.47656943_47656949delinsCCCAGA, g.47656948dup, g.47656996dup, g.47657000_47657001dup, g.47630449del, g.47657007dup, g.47657008del, g.47657020_47657023dup, g.47657025_47657026del, g.47657026dup, g.47657030_47657031del, g.47657047_47657050del, g.47657053del, g.47657053_47657057del, g.47657064del, g.47657073dup, g.47657312_47676594del, g.47668611_47674615del, g.47672116_47675123del, g.47666463_47677632del, g.47666403_47677572del, g.(47657081_47672686)_(47710367_?)del, g.(47657081_47672686)_(47710367_?)inv, g.47671507_47675022delinsCATTCTCTTTGAAAA, g.47657278_47676557del, g.47672687_47672796del, g.(47657081_47672686)_(47672797_47690169)del, g.(47657081_47672686)_(47672797_47690169)del, g.(47657081_47672686)_(47693948_47698103)del, g.(47657081_47672686)_(47698202_47702163)del, g.(47657081_47672686)_(47708011_47709917)del, g.47672691dup, g.47672697dup, g.47672721_47672744delins47672748_47672771inv, g.47672728_47672729del, g.47672731dup, g.47672750_47672751insGG, g.47672755_47672758del, g.47672762_47672763del, g.47630466_47630494del, g.47686194_47697740del, g.(47672797_47690169)_(47710367_?)del, g.(47672797_47690169)_(47690294_47693796)del, g.(47672797_47690169)_(47693948_47698103)del, g.47690170_47693947del, g.(47672797_47690169)_(47693948_47698103)del, g.(47672797_47690169)_(47693948_47698103)dup, g.(47672797_47690169)_(47705659_47707834)del, g.47690173del, g.47690191del, g.47690216_47690217dup, g.47690227del, g.47690227dup, g.47690228_47690232del, g.47690230_47690231del, g.47690240del, g.47690240_47690243del, g.47630475del, g.47630475_47630476del, g.47690259_47690260delinsCT, g.47690277dup, g.47690280del, g.47690283dup, g.(47690294_47693796)_(47702410_47703505)del, g.47630484_47630485insG, g.47693838_47693839del, g.47693862del, g.47693864del, g.47693873del, g.47693880dup, g.47693913del, g.47693924_47693925dup, g.47630493del, g.47697730_47706125del, g.(47693948_47698103)_(47710367_?)del, g.(47693948_47698103)_(47698202_47702163)del, g.(47693948_47698103)_(47705659_47707834)del, g.47698107del, g.47698109del, g.47698109_47698110insA, g.47630496del, g.47698118del, g.47698125del, g.47698129dup, g.47698138_47698139del, g.47698142_47698146del, g.47698144dup, g.47698147_47698148del, g.47698147_47698148dup, g.47698147_47698148insT, g.47698159del, g.47698162del, g.47698506_47703472del, g.47701803_47708848del, g.(47698202_47702163)_(47710367_?)del, g.(47698202_47702163)_(47702410_47703505)del, g.(47698202_47702163)_(47703711_47705410)del, g.(47698202_47702163)_(47705659_47707834)del, g.47702164del, g.47702175_47702176insA, g.47702183_47702186del, g.47702185_47702186insCT, g.47702190_47702192del, g.47702191dup, g.47702192_47702193del, g.47702213del, g.47702231del, g.47702242dup, g.47702257del, g.47702262_47702263dup, g.47630516_47630517dup, g.47630517del, g.47630517dup, g.47702289_47702290inv, g.47702293_47702296del, g.47702301dup, g.47702315del, g.47702315del, g.47702328_47702329del, g.47630522dup, g.47702339del, g.47702371_47702374dup, g.47702384_47702385del, g.47702386_47702389del, g.47702388del, g.47702388_47702389del, g.47702390del, g.47702390_47702391del, g.47702400_47702401del, g.47703506_47703710del, g.47703506_47708010del, g.47703510del, g.47703515del, g.47703521_47703522del, g.47703535_47703536del, g.47703546_47703547del, g.47703548_47703611dup, g.47630534del, g.47703571dup, g.47703574_47703581del, g.47703585dup, g.47630350del, g.47632107_47668733del, g.47703613del, g.(47630542_47635539)_(47643569_47656880)del, g.(47630542_47635539)_(47643_569_47656880)inv, g.(47630542_47635539)_(47657081_47672686)del, g.47635540_47657080del, g.(47630542_47635539)_(47672797_47690169)del, g.(47630542_47635539)_(47690294_47693796)del, g.(47630542_47635539)_(47705659_47707834)del, g.47635540_47635694del, g.(47630542_47635539)_(4763_5695_47637232)del, g.(47630542_47635539)_(47635695_47637232)del, g.(47630542_47635539)_(47637512_47639552)del, g.47703635dup, g.47703641dup, g.47635542_47635549del, g.47703660_47703663del, g.47703667dup, g.47630351dup, g.47703704del, g.47703826_47707938del, g.(47703711_47705410)_(47705659_47707834)del, g.47705428_47705431del, g.47705437_47705438insA, g.47635551_47635552del, g.47705440_47705441del, g.47705461del, g.47705490del, g.47705494del, g.47705495del, g.47635557_47635558del, g.47705505del, g.47705535dup, g.47705547del, g.47705560_47705561dup, g.47705561dup, g.47705562dup, g.47705588del, g.47705608_47705609del, g.47705618dup, g.47705627dup, g.47635571_47635601delins(217), g.(47705659_47707834)_(47710367_?)del, g.(47705659_47707834)_(47708011_47709917)del, g.47707842_47707843del, g.47707861del, g.47707861_47707874dup, g.47707878_47707884del, g.47707878_47707884del, g.47707883del, g.47707895_47707905del, g.47707897del, g.47707901_47707902del, g.47707905_47707906del, g.47707921del, g.47635583dup, g.47635583_47635584del, g.47707969_47707973del, g.47707996_47707997ins(115), g.47708009_47708010del, g.(47708011_47709917)_(47710367_?)del, g.47635591_47635592del, g.47635597_47635618dup, g.47635606_47635607del, g.47630359dup, g.47635672del, g.47635675_47635678del, g.47630364dup, g.47635680dup, g.47636862_47639040del, g.47636781_47638831del, g.47636753_47638155del, g.47636552_47638597del, g.(47635695_47637232)_(47643569_47656880)del, g.(47635695_47637232)_(47643569_47656880)del, g.(47635695_47637232)_(47657081_47672686)del, g.(47635695_47637232)_(47672797_47690169)del, g.(47635695_47637232)_(47698202_47702163)del, g.(47635695_47637232)_(47637512_47639552)del, g.(47635695_47637232)_(47641558_47643434)del, g.47637234del, g.47637246_47637247del, g.47637253_47637254del, g.47637254_47637255del, g.47637254_47637255del, g.47637265del, g.47637274del, g.47637282del, g.47637320del, g.47637372_47637375del, g.47637377_47637449dup, g.47637379del, g.47637384del, g.47637394_47637395del, g.47637396_47637397del, g.47637417del, g.47637427_47637435del, g.47637437_47637439del, g.47637453del, g.47637458dup, g.47637479_47637482dup, g.47637482dup, g.47637504_47637505del, g.47637508_47637511del, g.47638050_47653430del, g.47638302_47648462del, g.47638478_47648643del, g.(47637512_47639552)_(47710367_?)del, g.(47637512_47639552)_(47643569_47656880)del, g.47639553_47643568del, g.(47637512_47639552)_(47657081_47672686)del, g.(47637512_47639552)_(47657081_47672686)del, g.(47637512_47639552)_(47672797_47690169)del, g.(47637512_47639552)_(47639700_47641407)del, g.(47637512_47639552)_(47641558_47643434)del, g.47639557_47639561del, g.47639582_47639586delinsTAAT, g.47639583_47639584del, g.47639594del, g.47639594dup, g.47639598del, g.47639603_47639604del, g.47639611_47639612del, g.47639612del, g.47639618_47639621del, g.47639624_47639628delinsTTA, g.47630401dup, g.47639632dup, g.47639638_47639641dup, g.47639638_47639641dup, g.47639639del, g.47639639del, g.47639642dup, g.47630403_47630404insC, g.47639653del, g.47639666del, g.47639666_47639669del, g.47639668del, g.47639670_47639673delinsTT, g.47639674_47639675dup, g.47639695_47639696del, g.47639707_47642985del, g.47641402_47642007del, g.(47639700_47641407)_(47643569_47656880)del, g.47641408_47643568del, g.(47639700_47641407)_(47657081_47672686)del, g.(47639700_47641407)_(47672797_47690169)del, g.(47639700_47641407)_(47641558_47643434)del, g.(47639700_47641407)_(47641558_47643434)del, g.47641410del, g.47641425_47641426del, g.47641426_47641429del, g.47630412del, g.47641451del, g.47641454dup, g.47641455dup, g.47641469del, g.47641478del, g.47641488_47641491del, g.47641496_47641497del, g.47641503del, g.47641513_47641514dup, g.47641530_47641537dup, g.47642509_47655432del, g.(47641558_47643434)_(47643569_47656880)del, g.(47641558_47643434)_(47693948_47698103)del, g.47630424_47630433del, g.47643450dup, g.47643462_47643463del, g.47643462_47643463ins(4), g.47643464_47643465insNC_000022.10:35788169_35788352, g.47643465dup.

MutS Homolog 3 (MSH3) is a human homologue of the bacterial mismatch repair protein MutS that participates in the mismatch repair (MMR) system. MSH3 typically forms the heterodimer MutSβ with MSH2 in order to correct long insertion/deletion loops and base-base mispairs in microsatellites during DNA synthesis. Deficient capacity for MMR is found in approximately 15% of colorectal cancers, and somatic mutations in the MSH3 gene can be found in nearly 50% of MMR-deficient colorectal cancers. Examples of the mutations in MSH3 include, but are not limited to, g.79970809del.

MSH6 encodes MutS homologue 6 (MSH6), a member of the Mutator S (MutS) family of proteins that are involved in DNA mismatch repair (MMR). The MSH6 protein forms a heterodimer with MutS homologue 2 (MSH2) in both human and yeast. Human MSH2/6 recognizes single base-base mismatches and short insertion/deletion loops. Upon recognition of a mismatch, MSH2/6 complex binds and exchanges ADP for ATP, resulting in a conformational change to the complex that precedes base pair dissolution, base excision, and repair.

MSH6 mutations include frameshift and/or nonsense mutations and can result in non-functional MSH6 and loss of protein expression. Examples include a frameshift mutation at MSH6 amino acid residue 290 and a compounding missense T1189I.

Inactivating MSH6 mutations can be detected in cancers by routine diagnostics methods. These methods include, but are not limited to, obtaining cancer cells and other diagnostic indicators such as peripheral blood mononuclear cells (PBMCs), PBMC subpopulations, circulating blasts (CD34+ cells), circulating tumor cells and circulating exosomes cancer cells by biopsy and blood tests and by obtaining lymphatic or other bodily fluids. It is then determined from the cancer cells or other diagnostic indicators whether the cancer exhibits an inactivating MSH6 mutation is by methodology known in the art, for example, direct DNA sequencing and multiplex ligation dependent probe amplification, RNA sequencing (RNA-Seq), microarray, quantitative PCR, or NanoString gene expression panels, or MSH6 protein by immunohistochemistry, flow cytometry, immunocytochemistry or Western blot. Methods for identifying inactivating MSH6 mutations are disclosed in Houlleberghs H, Goverde A, Lusseveld J, Dekker M, Bruno M J, et al. (2017) Suspected Lynch syndrome associated MSH6 variants: A functional assay to determine their pathogenicity. PLOS Genetics 13(5): e1006765. https://doi.org/10.1371/journal.pgen.1006765.

Examples of the mutations in MSH6 include, but are not limited to, g.48032846_48032849del, g.48032846_48032849del, g.48032846_48032849del, g.48033337_48033342del, g.48033420_48033422del, g.(?_48010221)_(48034092)del, g.(?_48010221)_(48018263_48023032)del, g.47998510_48020183del, g.48007276_48020272del, g.48026207del, g.48026223del, g.48026223del, g.48026257_48026261del, g.48026261_48026265del, g.48026312_48026313del, g.48026398del, g.48026543_48026544dup, g.48026693dup, g.48026702del, g.48026712del, g.48026718dup, g.48026736_48026737delinsAG, g.48026736_48026737delinsG, g.48026750_48026751del, g.48026754_48026757del, g.48026756_48026759del, g.48026759_48026760del, g.48026906del, g.48026928_48026931del, g.48026941dup, g.48026991del, g.48027023_48027024del, g.48027079del, g.48027079_48027082dup, g.48027167_48027168del, g.48027172_48027173dup, g.48027178_48027185del, g.48027184_48027185del, g.48027272_48027275del, g.48027470_48027471del, g.48027501_48027502del, g.48027501_48027502delTG, g.48027657dup, g.48027691_48027694del, g.48027733_48027736dup, g.48027794_48027796delinsC, g.48027841_48027842del, g.48027887del, g.48027890dup, g.48027973_48027980del, g.48028067del, g.48028098del, g.48028106del, g.48028175_48028176del, g.48028241_48028242del, g.48028241_48028242delTT, g.48028272_48028284dup, g.48028277_48028278del, g.48030558_48030559del, g.48030126_48032394del, g.48030568del, g.48030581_48030584del, g.48030584_48030585dup, g.48030607del, g.48030645_48030646insT, g.48030647del, g.48030647dup, g.48030649dup, g.48030654_48030660del, g.48030659dup, g.48030697_48030698del, g.48030698del, g.48030706del, g.48030710dup, g.48030727_48030728insC, g.48030765_48030829del, c.3438+797_3438+798insTATins1839_3439-428, c.3438+797 3438+798insTATins1839_3439-428, g.48032121_48032122del, g.48032123_48032124del, g.48032124dup, g.48032126_48032129del, g.48032129_48032130insA, g.48032129_48032132dup, g.(48032167_48032756)_(48034092_?)del, g.48032809_48032812del, g.48032835dup, g.48032846_48032849del, g.48033374_48033402dup, g.48033395_48033398del, g.48033421_48033433del, g.48033425_48033428dup, g.48033453_48033454insA, g.48033494_48033523del, g.48033495_48033496del, g.48033593dup, g.48033610_48033613dup, g.48033629_48033635del, g.48033636_48033639dup, g.48033676_48033682del, g.48033707dup, g.48033709_48033716dup, g.48033721_48033724dup, g.48033727_48033730dup, g.48033728_48033746dup, g.(48033742_48033743)_(48033742_48033743)ins(32), g.48033746dup, g.48033748_48033751del, g.48033758_48033768del, g.48033773_48033774insATCA, g.48033773_48033776dup, g.48033785_48033789dup, g.48033887_48033910inv, g.(48018263_48023032)_(48032167_48032756)del, g.(48018263_48023032)_(48023203_48025749)del, g.48023097_48023098del, g.48025773dup, g.48025832del, g.48025860_48025861insT, g.48025884_48025885del, g.48025967dup.

MutL homolog 1, colon cancer, nonpolyposis type 2 (E. coli) is a protein that in humans is encoded by the MLH1 gene located on Chromosome 3. It is a gene commonly associated with hereditary nonpolyposis colorectal cancer.

Examples of the mutations in MSH6 include, but are not limited to, g.37089113_37089115del, g.37089175del, g.37090379_37090393del, g.37038201_37038202del, g.37042531_37042542del, g.37053339_37053355del, g.37053354del, g.37053590_37053591insT, g.37034841_37092337del, g.(?_37034841)_(37092337_?)del, g.(?_37034841)_(37061955_37067127)del, g.(?_37034841)_(37035155_37038109)del, g.(?_37034841)_(37035155_37038109)del, g.(?_37034841)_(37070424_37081676)del, g.(?_37034841)_(37083823_37089009)del, g.37034841_37083822del, g.(?_37034841)_(37038201_37042445)del, g.(?_37034841)_(37042545_37045891)del, g.37034841_37042544del, g.(?_37034841)_(37042545_37045891)del, g.(?_37034841)_(37042545_37045891)del, g.(?_37034841)_(37045966_37048481)del, g.(?_37034841)_(37050397_37053310)del, g.(?_37034841)_(37059091_37061800)del, g.37034658_37038806del, g.36961079_37138741del, g.37061923del, g.37061927del, g.37061933del, g.37061939del, g.37061942dup, g.37035140_37035141del, g.37070417del, g.37070417_37070418insT, g.37070419dup, g.37070422_37070423insT, g.37080355_37083368del, g.(37070424_37081676)_(37092337_?)del, g.(37070424_37081676)_(37081786_37083758)del, g.(37070424_37081676)_(37083823_37089009)del, g.37038148_37038151del, g.37038149del, g.37038149dup, g.37081690_37081691del, g.37081691_37081692del, g.37081706_37081708del, g.37081710_37081711del, g.37035053_37035066del, g.37038154del, g.37038154_37038157del, g.37081738_37081739del, g.37081740del, g.37081753dup, g.37081757_37081761dup, g.37081782_37081783insAAGT, g.37081787_37081793delinsATTT, g.(37081786_37083758)_(37083823_37089009)del, g.(37081786_37083758)_(37089175_37090007)del, g.37083759del, g.37083780dup, g.37083781_37083784del, g.37083781_37083784delCTCA, g.37083808_37083809del, g.37083816del, g.37086069_37089606del, g.37084092_37089247del, g.37084590_37089786del, g.(37083823_37089009)_(37092337_?)del, g.(37083823_37089009)_(37089175_37090007)del, g.37089010_37089174del, g.(37083823_37089009)_(37090509_37091976)del, g.37089023del, g.37089026_37089027del, g.37089027del, g.37089036del, g.37089036dup, g.37038168dup, g.37089042del, g.37089047del, g.37089050_37089053del, g.37089056_37089057del, g.37089061_37089062del, g.37089078_37089096del, g.37089090dup, g.37089099dup, g.37089107_37089110dup, g.37089109_37089110del, g.37089130_37089132del, g.37089130_37089132delAAG, g.37089131delinsTTCTT, g.37089133del, g.37089133delG, g.37089144del, g.37089155del, g.37089155_37089161del, g.37089158_37089161del, g.37089162_37089166del, g.37089171del, g.(37089175_37090007)_(37090101_37090394)del, g.37035056_37035072del, g.37090013del, g.37090015dup, g.37038183_37038184del, g.37090024_37090037dup, g.37090025_37090053dup, g.37090027dup, g.37038184dup, g. 37090031_37090032insT, g.37090041del, g.37090057del, g.37090064_37090067del, g.37038188del, g.37090082del, g.37090086_37090087del, g.37090087_37090088del, g.37090097_37090101delinsC, g.37090099del, g.37038191dup, g.(37090101_37090394)_(37092337_?)del, g.37035057_37035073del, g.37090405dup, g.37090411_37090415del, g.37090414del, g.37038194del, g.37038198del, g.37090472_37090478del, g.37039445_37059613dup, g.37039760_37052440del, g.37090481_37090482del, g.37090483_37090484del, g.37090483_37092045del, g.37040732_37043185delinsACATAGTA, g.37042445_37042446del, g.(37038201_37042445)_(37042545_37045891)del, g.(37038201_37042445)_(37048555_37050304)del, g.(37038201_37042445)_(37050397_37053310)del, g.(37038201_37042445)_(37053591_37055922)del, g.37090497_37090498del, g.37090497_37090498delTC, g.37090504_37090507del, g.(37090509_37091976)_(37092337_?)del, g.(37090509_37091976)_(37092337_?)dup, g.37091977_37091978del, g.37091978_37091987del, g.37042448_37042451del, g.37091984_37091990del, g.37042451_37042453del, g.37092020_37092021del, g.37092022_37092068dup, g.37092027_37092028del, g.37092027_37092028dup, g.37092030dup, g.37092052_37092055del, g.37092054_37092055del, g.37092068_37092071dup, g.37092091dup, g.37092094_37092097delins(30), g.37092096_37092106del, g.37092097del, g.37092125_37092126delAA, g.37092125_37092126del, g.37092139_37092142dup, g.37092142dup, g.37035060dup, g. 37042469_37042470del, g.37042470del, g.37042482dup, g.37042485del, g.37042499del, g.37042546dup, g.37044472_37046589del, g.37045648_37049941del, g.37045095_37054651del, g.37045072_37046861del, g.(37042545_37045891)_(37045966_37048481)del, g.(37042545_37045891)_(37092337_?)del, g.(37042545_37045891)_(37048555_37050304)del, g.(37042545_37045891)_(37050397_37053310)del, g.37045892_37050396del, g.37035069del, g.37045926del, g.37045931del, g.37045939_37045940dup, g.37045957_37045958del, g.37045963del, g.37035075del, g.37048067_37049287del, g.(37045966_37048481)_(37048555_37050304)del, g.(37045966_37048481)_(37050397_37053310)del, g.37048483del, g.37048483_37048503delinsT, g.37048486_37048487delinsGTT, g.37048489del, g.37048490del, g.37035076_37035077insCCCA, g.37035077_37035078dup, g.37048505_37048508del, g.37048521del, g.37048529dup, g.37035082dup, g.37049873_37052281del, g.37049839_37052249del, g.37049800_37052209del, g.37049640_37050445del, g.37050305_37050396del, g.(37048555_37050304)_(37050397_37053310)del, g.37050305_37050396del, g.37050319_37050320del, g.37050339del, g.37050348del, g.37050353_37050354del, g.37050354dup, g.37050364del, g.37050375_37050376insGA, g.37035090del, g.37050382_37050383delinsAT, g.37050382_37050383delinsCT, g.37050390_37050396del, g.37052950_37060990del, g.(37050397_37053310)_(37067499_37070274)dup, g.(37050397_37053310)_(37053591_37055922)del, g.(37050397_37053310)_(37056036_37058996)del, g.37053353del, g.37053510_37053511del, g.37035099del, g.37053545_37053546insT, g.37053562del, g.37053578del, g.37053578dup, g.37053585del, g.37053586_37053589del, g.37053591del, g.37053590_37053591delinsAT, g.37055920_37055921del, g.37055914_37055938del, g.(37053591_37055922)_(37070424_37081676)del, g.(37053591_37055922)_(37083823_37089009)del, g.(37053591_37055922)_(37059091_37061800)del, g.37035105del, g.37055928dup, g.37035106_37035116del, g.37055938del, g.37035108del, g.37055972_37055975del, g.37055976_37055979del, g.37035111del, g.37055990dup, g.37035114del, g.37035116del, g.37056036del, g.37056037dup, g.37058993_37059001del, g.(37056036_37058996)_(37070424_37081676)del, g.(37056036_37058996)_(37059091_37061800)del, g.37058997_37059000del, g.37059014_37059017del, g.37059017_37059021del, g.37059027_37059030dup, g.37035122del, g.37059062_37059063insT, g.37059065_37059066del, g.37059066del, g.37059066dup, g.37059072_37059073del, g.37059072_37059073dup, g.37059090_37059093del, g.37061595_37061913del, g.37061308_37066756del, g.37061207_37063077del, g.(37059091_37061800)_(37092337_?)del, g.(37059091_37061800)_(37061955_37067127)del, g.37061801_37061954del, g.(37059091_37061800)_(37083823_37089009)del, g.37061803dup, g.37061804del, g.37061817del, g.37061837_37061838dup, g.37061844del, g.37061851dup, g.37061855dup, g.37061870del, g.37061904_37061906del, g.37061910del, g.37035047del, g. [37049179_37051317delinsTG; 37051667_37054327delinsCA].

Human PMS2 related genes are located at bands 7p12, 7p13, 7q11, and 7q22. Exons 1 through 5 of these homologues share high degree of identity to human PMS2. The product of this gene is involved in DNA mismatch repair. The protein forms a heterodimer with MLH1 and this complex interacts with MSH2 bound to mismatched bases. Defects in this gene are associated with hereditary nonpolyposis colorectal cancer, with Turcot syndrome, and are a cause of supratentorial primitive neuroectodermal tumors.

Examples of the mutations in PMS2 include, but are not limited to, g.(?_6012870)_(6048737_?)del, g.6012870_6048737del, g.(6027252_6029430)_(6048737_?)del, g.(6045663_6048627)_(6048737_?)del, g.6029554del, g.6029499dup, g.6029495_6029496del, g.6029462_6029463delinsTAAA, g.5992485_60 28601del, g.(6018328_6022454)_(6027252_6029430)del, g.(6013174_6017218)_(6027252_6029430)del, g. 6027226_6027227in5(20), g.6027175del, g.6027090dup, g.6036705_60 44207delinsCG, g.6026666dup, g.6026628del, g.6043671del, g.6026565dup, g.6026565dupT, g.6018315_60 18316del, g.6018306_6018310del, g.6018306_6018310delAGTTA, g.6043633_6043634dup, g.6018256_6018259del, g.6015623_6017501del, g.6016429_6017479del, g.6017300_6017303del, g.6045579_6045674delinsATTT, g.(6043690_6045522)_(6045663_6048627)del, g.(?_6012870)_(6042268_6043320)del, g.(6035265_60 36956)_(6042268_6043320)del, g.6038283_6039384del, g.6038901del, g.6038851dup, g.(6035265_60 36956)_(6037055_60 38738)del, g.6037019_6037024delinsCTTCACACACA, g.6036980del, g.6036958dup, g.6035323_6035324insJN866832.1, g.(6022623_6026389)_(6035265_60 36956)del, g.(6031689_6035164)_(6035265_60 36956)del, g. 6035204_6035207del, g.6035205_60 35206del, g.(?_6012870)_(6031689_6035164)del, g.(6027252_6029430)_(6031689_6035164)del, g.(6029587_6031603)_(6031689_6035164)del, g.6028725_60 29882del, g.(?_6012870)_(6029587_6031603)del.

The present disclosure provides a method of treating patients with Lynch syndrome to reduce the likelihood of from developing or treating cancers derived from Lynch syndrome, by administering to the subject an effective amount of one or more disclosed compounds, or a pharmaceutically acceptable salt thereof, or the corresponding pharmaceutical composition.

Lynch syndrome is a hereditary disorder caused by a mutation in a mismatch repair gene in which affected individuals have a higher than normal chance of developing colorectal cancer, endometrial cancer, and various other types of aggressive cancers, often at a young age—also called hereditary nonpolyposis colon cancer (HNPCC).

The mutations of specific mismatch repair (MMR) genes including but not limited to MLH1, MSH2, MSH6, PMS2, and EPCAM-TACSTD1 deletions are responsible for Lynch syndrome. These genes work in repairing mistakes made when DNA is copied in preparation for cell division. The defects in the genes disallow repair of DNA mistakes and as cells divide, errors stack and uncontrollable cell growth may result in cancer.

Those with Lynch syndrome carry up to an 85% risk of contracting colon cancer as well as a higher than average risk for endometrial cancer, stomach cancer, pancreatic cancer, kidney/ureter tract cancer, hepatobiliary tract cancer, gastric tract cancer, prostate cancer, ovarian cancer, gallbladder duct cancer, brain cancer, small intestine cancer, breast cancer, and skin cancer.

In some embodiments for the disclosed method, the method is a method of treating cancer derived from Lynch syndrome, selected from the group consisting of colon cancer, endometrial cancer, stomach cancer, pancreatic cancer, kidney/ureter tract cancer, hepatobiliary tract cancer, gastric tract cancer, prostate cancer, ovarian cancer, gallbladder duct cancer, brain cancer, small intestine cancer, breast cancer, and skin cancer.

In some embodiments, the method is a method of treating autoimmune disease. Exemplary autoimmune diseases include lupus erythematosus; Wiskott-Aldrich syndrome; autoimmune lymphoproliferative syndrome; myasthenia gravis; rheumatoid arthritis (RA); lupus nephritis; multiple sclerosis; systemic lupus erythematosis; discoid lupus; subacute cutaneous lupus erythematosus; cutaneous lupus erythematosus including chilblain lupus erythematosus; chronic arthritis; Sjogren's syndrome; inflammatory chronic rhinosinusitis; colitis; celiac disease; inflammatory bowel disease; Barrett's esophagus; inflammatory gastritis; autoimmune nephritis; autoimmune vasculitis; autoimmune hepatitis; autoimmune carditis; autoimmune encephalitis; autoimmune diabetes; autoimmune diabetes nephritis; psoriasis; Graft-versus-host disease (GvHD); and autoimmune mediated hematological disease.

In some embodiments, the method is a method of treating immune deficiency selected from the group consisting of Autoimmune Lymphoproliferative Syndrome (ALPS), Autoimmune polyglandular syndrome type 1 (APS-1), BENTA Disease, Caspase Eight Deficiency State (CEDS), Chronic Granulomatous Disease (CGD), Common Variable Immunodeficiency (CVID), Congenital Neutropenia Syndromes, CTLA4 Deficiency, DOCK8 Deficiency, GATA2 Deficiency, Glycosylation Disorders With Immunodeficiency, hyper-immunoglobulin E syndrome (HIES), Hyper-Immunoglobulin M (Hyper-IgM) Syndromes, Leukocyte adhesion deficiency (LAD), LRBA deficiency, PI3 Kinase disease, PLCG2-associated antibody deficiency and immune dysregulation (PLAID), severe combined immunodeficiency (SCID), STAT3 gain-of-function disease, Warts, Hypogammaglobulinemia, Infections, and Myelokathexis Syndrome (WHIMS), X-Linked Agammaglobulinemia (XLA), X-Linked Lymphoproliferative Disease (XLP), and XMEN Disease.

As used herein, the term “immune deficiency” refers to a condition in which a portion or some portions of cell components constituting an immune system are defective or dysfunction, so that a normal immune mechanism is damaged. In other words, “immune deficiency” means a condition under which: congenital immunity and/or acquired immunity are suppressed and/or decreased. In some embodiments, the immune-deficiency subject is an immunocompromised subject. Non-limiting examples of immune deficiencies can include AIDS, hypogammaglobulinemia, agammaglobulinemia, granulocyte deficiency, chronic granulomatous disease, asplenia, SCID, complement deficiency, and/or sickle cell anemia.

In some embodiments, the method is a method of treating a neurodegenerative disorder selected from the group consisting of multiple sclerosis, Parkinson's disease (PD), Alzheimer's disease (AD), Dentatorubropallidoluysian atrophy (DRPLA), Huntington's Disease (HD), Spinocerebellar ataxia Type 1 (SCA1), Spinocerebellar ataxia Type 2 (SCA2), Spinocerebellar ataxia Type 3 (SCA3), Spinocerebellar ataxia 6 (SCA6), Spinocerebellar ataxia Type 7 (SCAT), Spinocerebellar ataxia Type 8 (SCAB), Spinocerebellar ataxia Type 12 (SCA12), Spinocerebellar ataxia Type 17 (SCA17), Spinobulbar Muscular Ataxia/Kennedy Disease (SBMA), Fargile X syndrome (FRAXA), Fragile XE mental retardation (FRAXE), and Myotonic dystrophy (DM).

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

In some embodiments, the methods disclosed herein further comprise co-administering an effective amount of a DNA repair inhibitor, a DNA damage response (DDR) inhibitor, a DNA damaging agent or an immunomodulatory agent to the subject being treated for cancer, in addition to an effective amount of a disclosed RAD51 inhibitor.

The term “DNA repair inhibitor” refers to any agent that targets components/processes which a cell uses to repair mutations or changes in DNA and restore the DNA to its original state and prevents the repair of DNA. Examples of DNA repair inhibitors include: RPA inhibitors, APE1 inhibitors, DNA ligase inhibitors, DNA polymerase inhibitors, Parp inhibitors etc.

The term “DNA damage response inhibitor” refers to any agent that targets components/processes involved in detecting DNA lesions, signaling the presence of DNA damage, and/or promote the repair of DNA damage. Examples of DNA damage response inhibitors include checkpoint inhibitors, ATM and ATR inhibitors, DNA-PK inhibitors, etc.

The term “DNA damaging agent” refers to any agent that directly or indirectly damages DNA for which homologous recombination could repair the damage. The DNA damaging agents is selected from the group consisting of: exposure to a DNA damaging chemical; exposure to a chemotherapeutic agent; exposure to a radiochemotherapy, and exposure to ionizing or ultraviolet radiation. Some examples of DNA-damaging chemotherapeutic agents include alkylating agents, nitrosoureas, anti-metabolites, plant alkaloids, plant extracts and radioisotopes. Some examples of the chemotherapeutic agents also include DNA-damaging drugs, for example, 5-fluorouracil (5-FU), capecitabine, S-1 (Tegafur, 5-chloro-2,4-dihydroxypyridine and oxonic acid), 5-ethynyluracil, arabinosyl cytosine (ara-C), 5-azacytidine (5-AC), 2′, 2′-difluoro-2′-deoxycytidine (dFdC), purine antimetabolites (mercaptopurine, azathiopurine, thioguanine), gemcitabine hydrochlorine (Gemzar), pentostatin, allopurinol, 2-fluoro-arabinosyl-adenine (2F-ara-A), hydroxyurea, sulfur mustard (bischloroetyhylsulfide), mechlorethamine, melphalan, chlorambucil, cyclophosphamide, ifosfamide, thiotepa, AZQ, mitomycin C, dianhydrogalactitol, dibromoducitol, alkyl sulfonate (busulfan), nitrosoureas (BCNU, CCNU, 4-methyl CCNU or ACNU), procarbazine, decarbazine, rebeccamycin, anthracyclins such as doxorubicin (adriamycin; ADR), daunorubicin (Cerubicine), idarubicin (Idamycin) and epirubicin (Ellence), anthracyclin analogs such as mitoxantrone, actinomycin D, non-intercalating topoisomerase inhibitors such as epipodophyllotoxins (etoposide or VP16, teniposide or VM-26), podophylotoxin, bleomycin (Bleo), pepleomycin, compounds that form adducts with nucleic acid including platinum derivatives, e.g., cisplatin (CDDP), trans analog of cisplatin, carboplatin, iproplatin, tetraplatin and oxaliplatin, as well as camptothecin, topotecan, irinotecan (CPT-11), and SN-38. Some examples of nucleic acid damaging treatments include radiation e.g., ultraviolet (UV), infrared (IR), or .alpha.-, .beta.-, or .gamma.-radiation, as well as environmental shock, e.g., hyperthermia.

“Immunomodulatory agent” means an agent that modulates an immune response to an antigen but is not the antigen or derived from the antigen. “Modulate”, as used herein, refers to inducing, enhancing, suppressing, directing, or redirecting an immune response. Such agents include immunostimulatory agents, such as adjuvants, that stimulate (or boost) an immune response to an antigen but is not an antigen or derived from an antigen. There are several distinct types of immunomodulatory agents, which include, but are not limited to, Toll-like Receptor (TLR) agonists and Toll-like Receptor (TLR) antagonists. Such agents also include immunosuppressants. The immunomodulatory agent is selected from the group consisting of immune checkpoint modulators, Toll-like receptor (TLR) agonists, cell-based therapies, cytokines and cancer vaccines.

In some embodiments, the subject is determined to have an increased level and/or activity of a DNA damage process or DNA editing enzyme. In one aspect of this embodiment, the DNA editing enzyme is selected from the group consisting of activation induced cytidine deaminase (AID or AICDA), APOBEC2, APOBEC3A, APOBEC3C, APOBEC3D, APOBEC3F, APOBEC3G, APOBEC3H, APOBEC4, a Type 1 Topoisomerase, a Type 2 Topoisomerase, Recombination Activating Gene 1 (RAG 1), and Recombination Activating Gene 2 (RAG2).

In some embodiments, blood cells obtained from the subject have been determined to have a detectable level of activation-induced cytidine deaminase (AID).

In some embodiments, B cells obtained from the subject have been determined to have a detectable level of activation-induced cytidine deaminase (AID).

In some embodiments, the detectable level of activation-induced cytidine deaminase (AID) is statistically significantly higher than the level of AID expressed in unactivated B-cells or normal non-immune cells from a healthy subject.

### Methods of Administration and Dosage Forms

The precise amount of compound administered to provide an “effective amount” to the subject will depend on the mode of administration, the type, and severity of the disease, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the disclosure being used by following, for example, dosages reported in the literature and recommended in the Physician's Desk Reference (57th ed., 2003).

The term “effective amount” means an amount when administered to the subject which results in beneficial or desired results, including clinical results, e.g., inhibits, suppresses or reduces the symptoms of the condition being treated in the subject as compared to a control. For example, a therapeutically effective amount can be given in unit dosage form (e.g., 0.1 mg to about 50 g per day, alternatively from 1 mg to about 5 grams per day).

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

In addition, the disclosed RAD51 inhibitors can be co-administered with other therapeutic agents. As used herein, the terms “co-administration”, “administered in combination with”, and their grammatical equivalents, are meant to encompass administration of two or more therapeutic agents to a single subject, and are intended to include treatment regimens in which the agents are administered by the same or different route of administration or at the same or different times. In some embodiments the one or more compounds described herein will be co-administered with other agents. These terms encompass administration of two or more agents to the subject so that both agents and/or their metabolites are present in the subject at the same time. They include simultaneous administration in separate compositions, administration at different times in separate compositions, and/or administration in a composition in which both agents are present. Thus, in some embodiments, the compounds described herein and the other agent(s) are administered in a single composition. In some embodiments, the compounds described herein and the other agent(s) are admixed in the composition.

The particular mode of administration and the dosage regimen will be selected by the attending clinician, taking into account the particulars of the case (e.g., the subject, the disease, the disease state involved, the particular treatment). Treatment can involve daily or multi-daily or less than daily (such as weekly or monthly etc.) doses over a period of a few days to months, or even years. However, a person of ordinary skill in the art would immediately recognize appropriate and/or equivalent doses looking at dosages of approved compositions for treating a RAD51 mediated disease using the disclosed RAD51 inhibitors for guidance.

The compounds or the corresponding pharmaceutical compositions taught herein can be administered to a patient in a variety of forms depending on the selected route of administration, as will be understood by those skilled in the art. The compounds of the present teachings may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time.

The pharmaceutical composition of the disclosure is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for intravenous administration.

Typically, for oral therapeutic administration, a compound of the present teachings may be incorporated with excipient and used in the form of ingestible tablets, buccal tablets, troches, capsules, elixirs, suspensions, syrups, wafers, and the like.

Typically for parenteral administration, solutions of a compound of the present teachings can generally be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms.

Typically, for injectable use, sterile aqueous solutions or dispersion of, and sterile powders of, a compound described herein for the extemporaneous preparation of sterile injectable solutions or dispersions are appropriate.

### EXAMPLES

### Example 1. Exposure of Compound 67A in Toxicology Species and Human Subjects

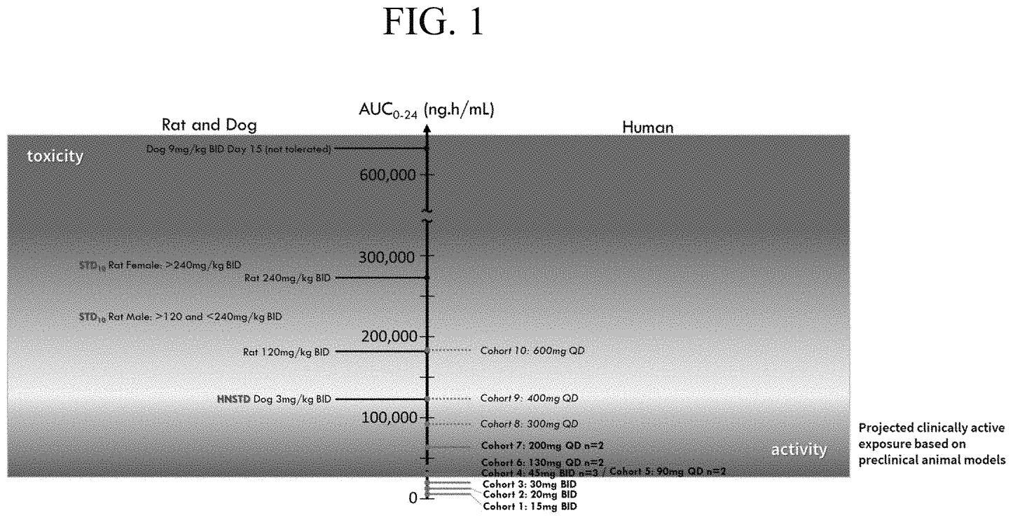

Compound 67A was administered to rat and dog for 28 days to assess toxicology findings (FIG. 1). These exposures in the toxicology species are compared with human subjects at the respective dose levels.

### Example 2. Compound 67A Pharmacokinetics

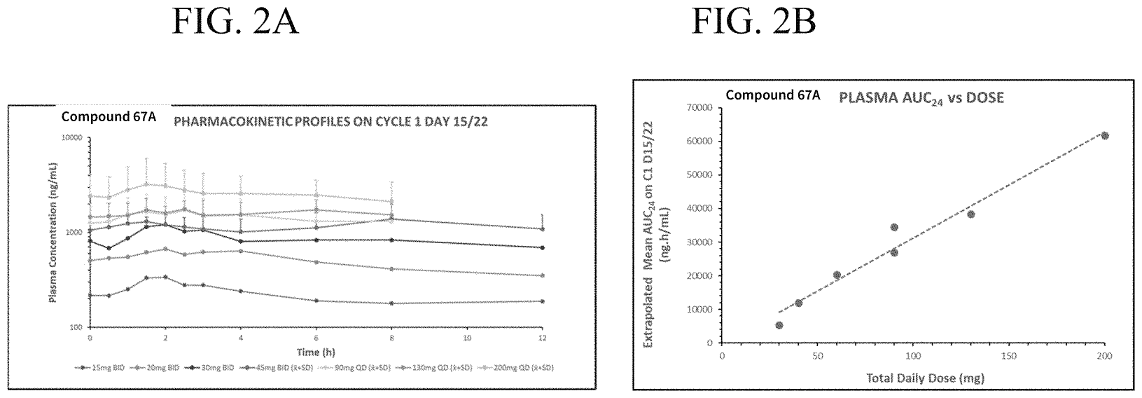

Compound 67A pharmacokinetics were analyzed in human subjects (Table 1, FIGS. 2A-2B)

TABLE 1

| Dose | | Cₘₐₓ | AUC₀₋ₜ |
|---|---|---|---|
| (mg) | Tₘₐₓ (h) | (ng/mL) | (ng · h/mL) |
| 15 BID | 2.0 | 338 | 2630^{c} |
| 20 BID | 2.0 | 670 | 5940^{c} |
| 30 BID | 2.0 | 1210 | 10200^{c} |
| 45 BID | 1.5 (1.5, 6)^{a} | 1430 ± 362^{b} | 13500 ± 8270^{b,c,d} |
| 90 QD | 2.5 (1, 2.5)^{a} | 1740 ± 930^{b} | 11500 ± 5570^{b,e} |
| 130 QD | 2.0 (1.5, 2.5)^{a} | 1800 ± 438^{b} | 12800 ± 3980^{b,e} |
| 200 QD | 3.75 (1.5, 6)^{a} | 3460 ± 2470^{b} | 20600 ± 12200^{b,e} |

^{a}median (min, max);
^{b}mean ± standard deviation (SD);
^{c}AUC₀₋₁₂;
^{d}n = 2;
^{e}AUC₀₋₈

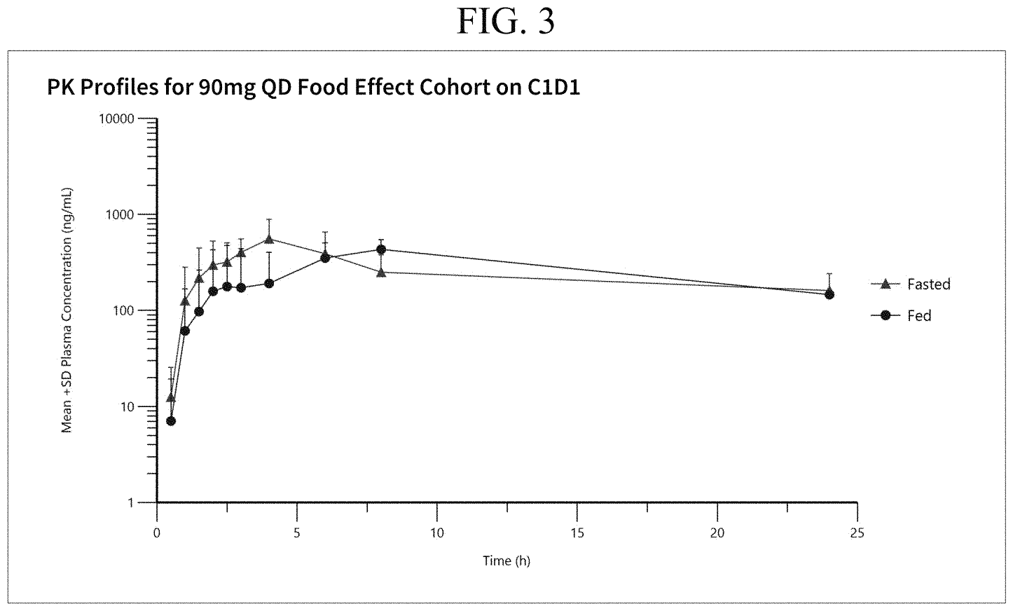

The food effect was evaluated with a 90 mg QD dosage during cycle 1 at day 1 (Table 2; FIG. 3). Subjects in the 90 mg QD food-effect cohort showed minimal impact of food on drug exposure.

TABLE 2

| | | |
|---|---|---|
| | Fasted Patients | Fed Patients |
| Pharmacokinetic | (n = 3) | (n = 3) |

| | |
|---|---|
| Parameter | Mean ± SD |

| | | |
|---|---|---|
| Tₘₐₓ (h) | 4 (2.5, 4)^{a} | 8 (6, 8)^{a} |
| Cₘₐₓ (ng/mL) | 574 ± 304 | 458 ± 129 |
| AUC₀₋₂₄ | 5950 ± 2900 | 6434 ± 1835 |
| (ng · h/mL) | | |

^{a}median (min, max)

### Example 3. Compound 67A Phase 1/2 Monotherapy

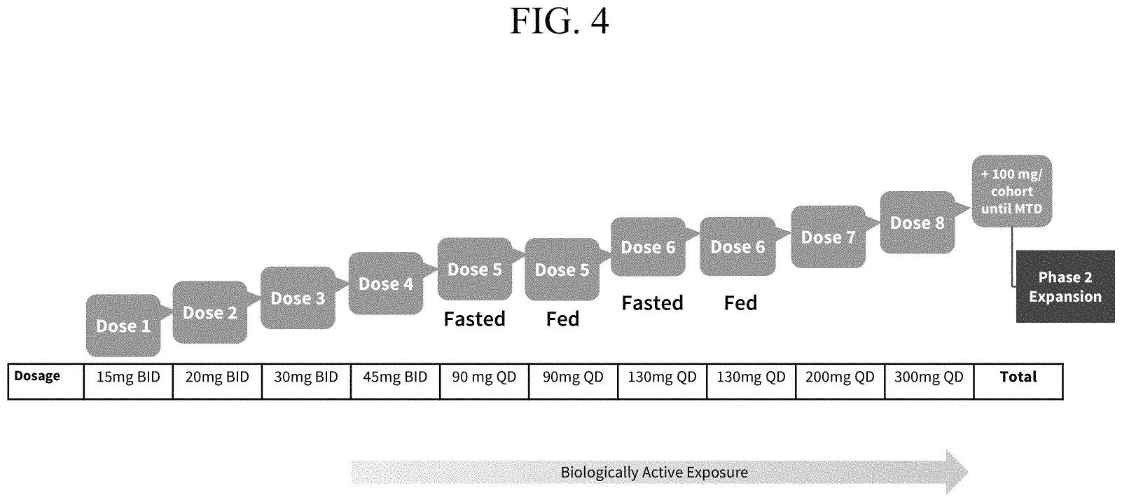

Compound 67A was analyzed in hematological and solid tumors. Dose escalation studies comprised of subjects with B-cell non-Hodgkin's lymphoma, chronic lymphocytic leukemia, multiple myeloma, breast cancer, head and neck cancer, soft tissue sarcoma, and ovarian cancer. Backfill studies comprised of subjects with diffuse large b-cell lymphoma (DLBCL), B-cell malignancies, pancreatic cancer, small-cell lung cancer, and HPV+HNSCC. Dosage for the Phase 1/2 monotherapy study is seen in FIG. 4.

Subjects with advanced hematologic and solid tumors were treated with continuous 28-day cycles of increasing doses of Compound 67A with an accelerated titration and 3+3 trial design. As of Dec. 8, 2020 twenty-three subjects were observed with advanced cancers (sarcoma n=8; breast cancer n=4; Non-Hodgkin's Lymphoma n=5; pancreatic cancer n=3; ovarian cancer n=2; and other n=1) were enrolled in 6 cohorts (15 mg, 20 mg, 30 mg, and 45 mg BID; 90 mg and 130 mg QD). No subjects experienced a dose-limiting toxicity and escalation continued per protocol to identify the MTD. Six subjects (26.1%) experienced a Compound 67A-related adverse event with only Grade 1/2 nausea (n=3, 13%) and constipation (n=2, 8.7%) occurring in >1 subject. There were no reported Compound 67A-related myelosuppression, serious adverse events, study discontinuation, or deaths. Preliminary pharmacokinetic analyses showed dose proportional systemic exposure with a half-life of −3 days supporting transition from BID to QD dosing.

Ten subjects were response evaluable prior to the data cut off. Two partial responses by Lugano and RECIST v1.1 criterion were achieved in subjects with DLBCL (−74%) and myxofibrosarcoma (−30%) at 45 mg BID with treatment ongoing at 126+ and 250+ days. An additional two subjects, with pancreatic cancer (−19%) and follicular lymphoma (−42%) had stable disease with tumor shrinkage at 45 mg BID for 111 and 99+ days.

### Clinical Efficacy

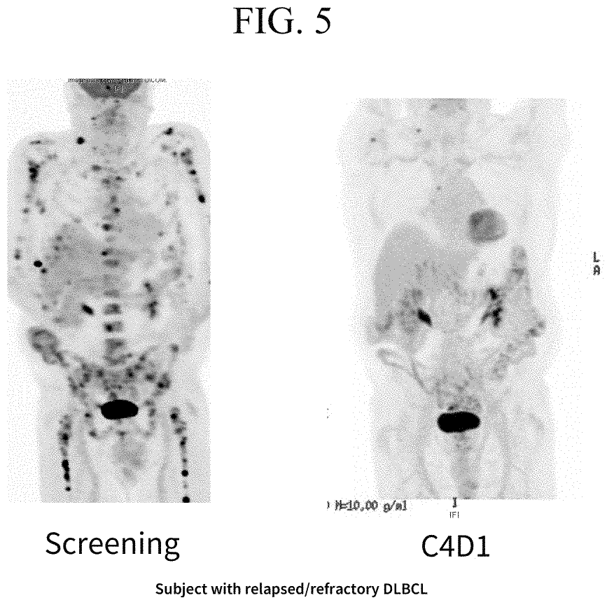

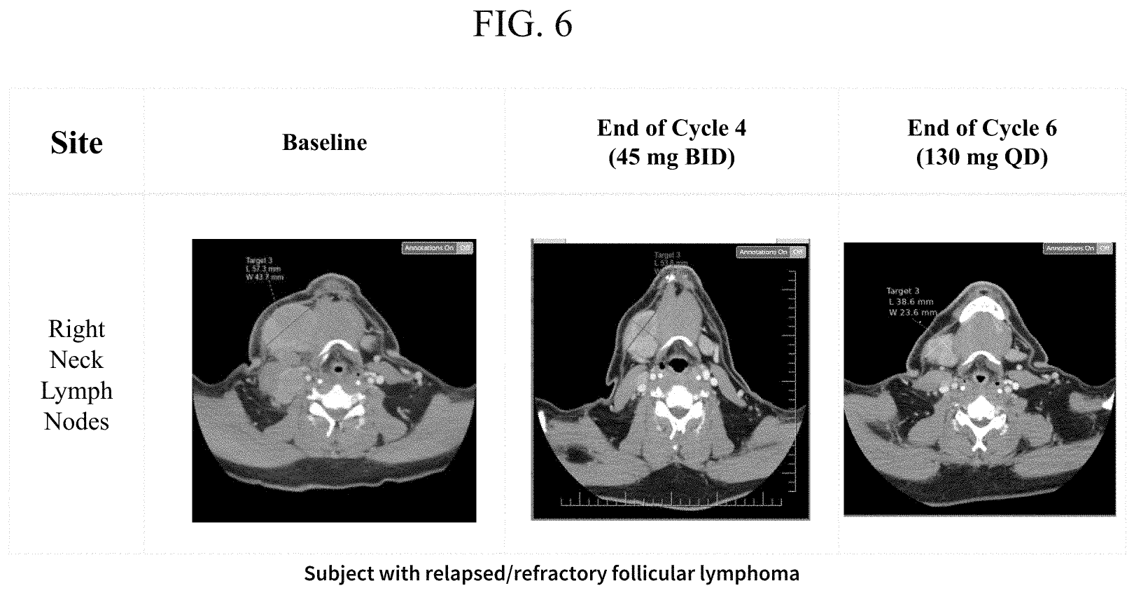

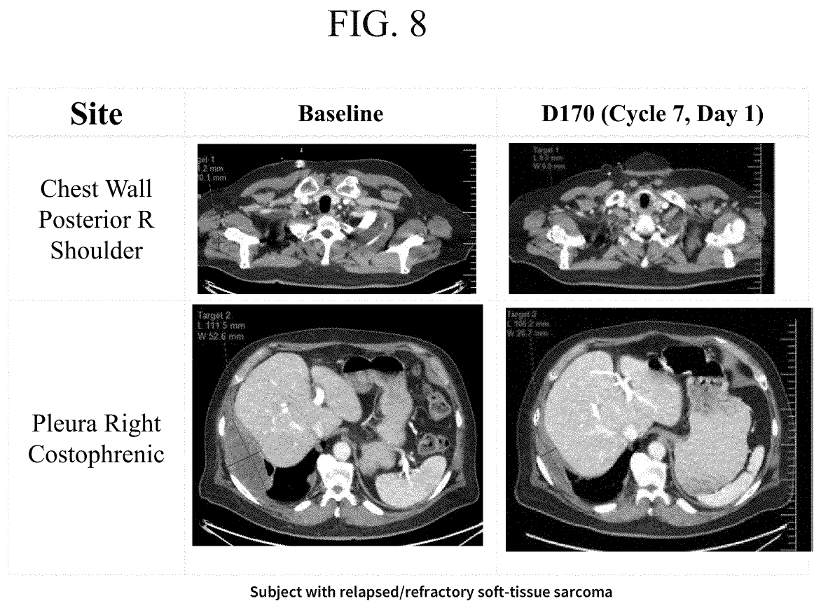

One subject with DLBCL showed a confirmed partial response with near complete resolution of target lesions and significant reduction in metabolic activity in non-target lesions and continued treatment after the third month (FIG. 5). One subject with myxofibrosarcoma showed an unconfirmed partial response in a patient with 30% decrease and continued treatment past the eleventh month (FIG. 8). One subject with follicular lymphoma showed an unconfirmed partial response at the end of the sixth month with a decrease of 42% at C3D1, 47% at C5D1, and dose escalated to 130 mg QD at C5D1 and then had an overall 67% at C7D1 and continued treatment (FIG. 6).

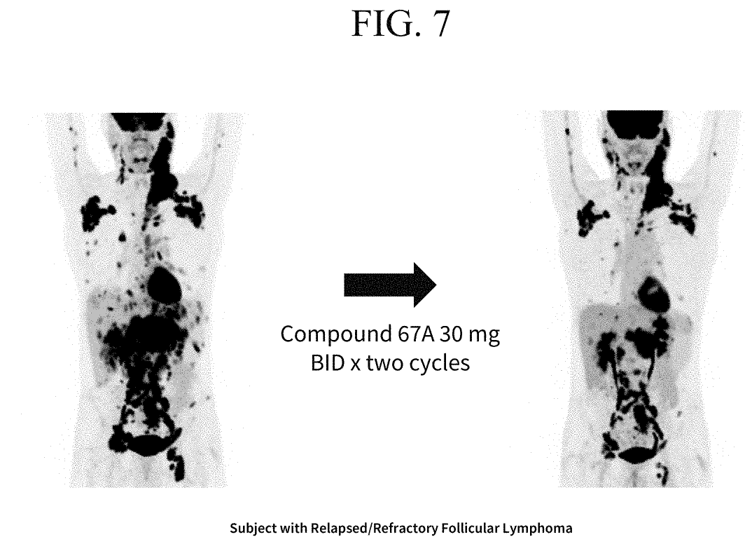

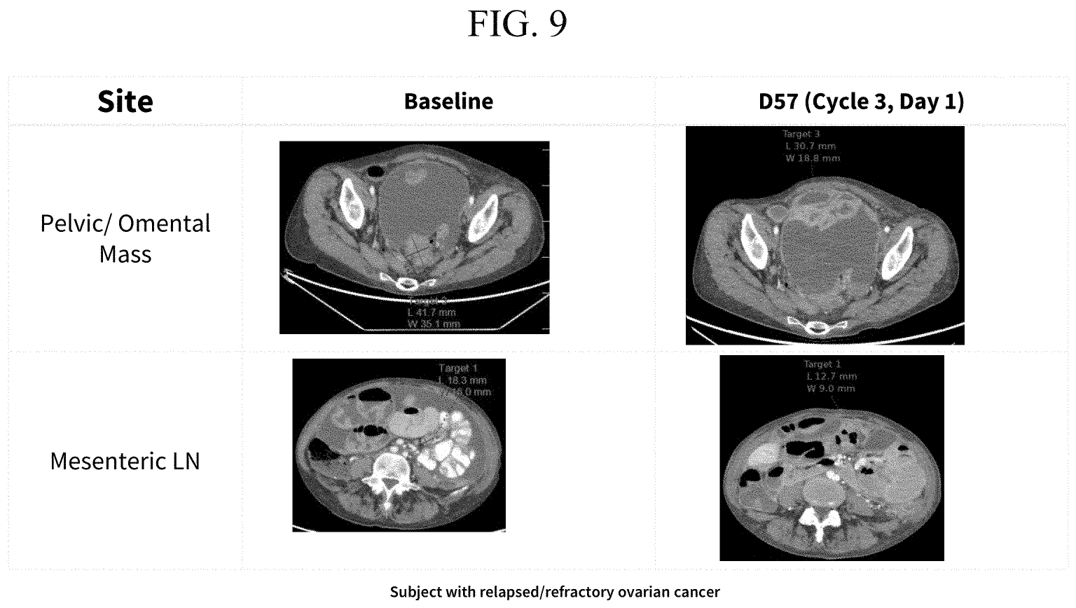

Of the four subjects that experienced stable disease with evidence of clinical benefit, one subject with follicular lymphoma showed a decrease of 27% at C3D1 (at 30 mg BID) and discontinued in month three for a new lesion (FIG. 7). One subject with pancreatic cancer showed a decrease of 19% by RECIST with concordant 70+% drop in CA19-9, progressed at C5D1. One subject with leiomyosarcoma showed stable disease with 0% by RECIST at six months and continued treatment past the eight month. One subject with ovarian cancer showed stable disease with a decrease of 29% by RECIST v1.1 at two months and continued treatment past the third month (FIG. 9).

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A method of treating a cancer comprising administering to a subject in need thereof Compound 67A 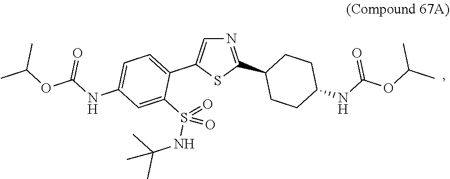 [SMILES: CC(C)OC(=O)Nc1ccc(-c2cnc([C@H]3CC[C@H](NC(=O)OC(C)C)CC3)s2)c(S(=O)(=O)NC(C)(C)C)c1]
or a pharmaceutically acceptable salt thereof, at a dosage from about 15 mg to about 700 mg.

2. The method of claim 1, wherein Compound 67A is administered at a dosage of about 15 mg.

3. The method of claim 1, wherein Compound 67A is administered at a dosage of about 20 mg.

4. The method of claim 1, wherein Compound 67A is administered at a dosage of about 30 mg.

5. The method of claim 1, wherein Compound 67A is administered at a dosage of about 45 mg.

6. The method of claim 1, wherein Compound 67A is administered at a dosage of about 90 mg.

7. The method of claim 1, wherein Compound 67A is administered at a dosage of about 130 mg.

8. The method of claim 1, wherein Compound 67A is administered at a dosage of about 200 mg.

9. The method of claim 1, wherein Compound 67A is administered at a dosage of about 300 mg.

10. The method of claim 1, wherein Compound 67A is administered at a dosage of about 400 mg.

11. The method of claim 1, wherein Compound 67A is administered at a dosage of about 500 mg.

12. The method of claim 1, wherein Compound 67A is administered at a dosage of about 600 mg.

13. The method of claim 1, wherein Compound 67A is administered at a dosage of about 700 mg.

14. The method of claim 1, wherein Compound 67A is administered at a dosage ranging from about 15 mg/day to about 1400 mg/day.

15. The method of claim 1, wherein Compound 67A is administered at a dosage ranging from about 90 mg to about 700 mg, about 100 mg to about 200 mg, about 100 mg to about 300 mg, about 100 mg to about 400 mg, about 100 mg to about 500 mg, about 100 mg to about 600 mg, about 100 mg to about 700 mg, about 200 mg to about 300 mg, about 200 mg to about 400 mg, about 200 mg to about 500 mg, about 200 mg to about 600 mg, about 200 mg to about 700 mg, about 300 mg to about 400 mg, about 300 mg to about 500 mg, about 300 mg to about 600 mg, about 300 mg to about 700 mg, about 400 mg to about 500 mg, about 400 mg to about 600 mg, about 400 mg to about 700 mg, about 500 mg to about 600 mg, about 500 mg to about 700 mg, and about 600 mg to about 700 mg.

16. The method of claim 1, wherein Compound 67A is administered at a dosage ranging from about 10 mg to about 50 mg, about 10 mg to about 40 mg, about 10 mg to about 30 mg, about 15 mg to about 50 mg, about 15 mg to about 40 mg, about 15 mg to about 30 mg, about 20 mg to about 50 mg, about 20 mg to about 40 mg, about 20 mg to about 30 mg, about 30 mg to about 50 mg, and about 30 mg to about 40 mg.

17. The method of any one of claims 1-16, wherein Compound 67A is administered once daily.

18. The method of any one of claims 1-16, wherein Compound 67A is administered twice daily.

19.-24. (canceled)

25. The method of claim 1, wherein the subject is human.

26. The method of claim 1, wherein the cancer is B-cell non-Hodgkin's lymphoma, chronic lymphocytic leukemia, multiple myeloma, breast cancer, head and neck cancer, soft tissue sarcoma, ovarian cancer, pancreatic cancer, follicular lymphoma, or mantle cell lymphoma.
